(19) United States
(12) Patent Application Publication
(10) Pub. No.: US 2019/0254904 A1
(43) Pub. Date: Aug. 22, 2019

(54) SUPPORT ARM SYSTEM, METHOD AND APPARATUS

(71) Applicant: Dave Schulz, La Grange, CA (US)

(72) Inventor: Dave Schulz, La Grange, CA (US)

(21) Appl. No.: 16/203,403

(22) Filed: Nov. 28, 2018

Related U.S. Application Data
(60) Provisional application No. 62/631,635, filed on Feb. 17, 2018.

(51) Int. Cl. A61G 13/12; A61D 3/00; A61F 5/37; A61G 7/075; A61G 13/10; F16M 11/12; F16M 13/02; F16M 13/04

(52) U.S. Cl. CPC A61G 13/1245; A61D 3/00; A61F 5/3761; A61G 7/0755; A61G 13/101; A61G 13/128; F16M 2200/06; F16M 13/022; F16M 13/04; A61G 2200/327; A61G 2203/70; F16M 2200/024; F16M 11/12

(57) ABSTRACT

A clamping system and method of clamping a rod includes a single knob coupled to a control screw. The single knob can increase or decrease a clamping force on the control screw. A clamp housing includes a control screw channel, wherein the control screw is disposed in the screw channel. A rod channel is capable of receiving a rod. A cleat channel extends into the rod channel. A cleat block has a clamping surface capable of entering at least a portion of the rod channel as the clamping force is increased on the cleat block.

### CROSS REFERENCE TO RELATED APPLICATIONS

This application claims priority from U.S. Provisional Patent Application No. 62/631,635 filed on Feb. 17, 2018 and entitled “Support Arm System, Method and Apparatus,” which is incorporated herein by reference in its entirety for all purposes.

### FIELD OF THE DISCLOSURE

The present disclosure relates generally to flexible, articulated support structures.

### BACKGROUND

In medical and other industries there exists a need for supporting workpieces in unique and varying positions and angles. One example is when a surgeon is performing surgery on a patient's limb. The patient's limb is unique, individual in size and shape and thus requires a support system that can be very easily and nearly infinitely adjustable to place and securely hold the patient's limb in an ideal position for the surgeon to perform the surgery. Many surgeons are limited by apparatus that cannot adjust easily and requires multiple hands and even multiple people to adjust or cannot securely hold the limb in the ideal position. There are other instances in other industries where the workpiece needs to be held in a unique position such as welding or other, similar situations for holding the workpiece.

It is in this context that the following embodiments arise.

### SUMMARY

Broadly speaking, the present disclosure fills these needs by providing an adjustable support arm system, method and apparatus. It should be appreciated that the present disclosure can be implemented in numerous ways, including as a process, an apparatus, a system or a device. Several inventive embodiments of the present disclosure are described below.

One implementation of the disclosed embodiments provides a clamping system that can clamp multiple rods or support arms in multiple angles using a single control knob. The clamp includes a clamping surface having a shape corresponding to the surface shape of the rod or rods being clamped. The clamping surface can move in a radial direction toward the center of the rod being clamped to provide clamping pressure directed toward the center of the rod being clamped.

Other aspects and advantages of the disclosure will become apparent from the following detailed description, taken in conjunction with the accompanying drawings, illustrating by way of example the principles of the disclosure.

### BRIEF DESCRIPTION OF THE DRAWINGS

The present disclosure will be readily understood by the following detailed description in conjunction with the accompanying drawings.

FIG. 1 is an illustration of the support arm in use mounted on a worktable, for implementing embodiments of the present disclosure.

FIG. 2 is another illustration of the support arm in use, mounted on a worktable, for implementing embodiments of the present disclosure.

FIGS. 3 and 4 are additional illustrations of the support arm in use mounted on the worktable, for implementing embodiments of the present disclosure.

FIGS. 5 and 6 are additional illustrations of the support arm supporting a horizontal rod that can be used to support a workpiece, for implementing embodiments of the present disclosure.

FIG. 7A is a simplified schematic diagram of several views of the support arm, for implementing embodiments of the present disclosure.

FIG. 7B is a schematic diagram of an exploded view of one implementation of the support arm, for implementing embodiments of the present disclosure.

FIG. 7C is another schematic diagram of another use of the support arm, for implementing embodiments of the present disclosure.

FIGS. 8A and 8B are exemplary detail views of corresponding implementations of the strut that could be used as part of the support arm system, for implementing embodiments of the present disclosure.

FIG. 9A provides multiple views of a single control knob, multiple orientation, clamp system that can be used with the support arm system or could be used in other implementations, independent of the support arm system, for implementing embodiments of the present disclosure.

FIGS. 9B and 9C are exploded views of the single knob, multiple orientation, clamp system, for implementing embodiments of the present disclosure.

FIGS. 9D and 9E are multiple views of the second cleat block, for implementing embodiments of the present disclosure.

FIG. 9F are multiple views of the friction system, for implementing embodiments of the present disclosure.

FIG. 9G provides multiple views of the first clamp housing, for implementing embodiments of the present disclosure.

FIG. 9G′ provides a progressive view of the cleat block moving into the cleat channel of the first clamp housing, for implementing embodiments of the present disclosure.

FIGS. 9H-J provide multiple views of the first cleat block, for implementing embodiments of the present disclosure.

FIGS. 9K and 9K′ illustrate the rounded clamping surface of the cleat block engaging a rounded surface of the rounded rod, for implementing embodiments of the present disclosure.

FIG. 9L provides multiple views of the second clamp housing, for implementing embodiments of the present disclosure.

FIG. 9M illustrates multiple views of a clamp housing engaging a rod having a rectangular cross-sectional shape, for implementing an embodiment of the present disclosure.

FIG. 9N illustrates multiple views of a clamp housing engaging a rod having a triangular cross-sectional shape, for implementing an embodiment of the present disclosure.

FIG. 10 provides multiple views of a clamp block of the base clamp, for implementing an embodiment of the present disclosure.

FIGS. 11A-11B provides multiple views of the base clamp housing, for implementing an embodiment of the present disclosure.

FIG. 12 provides multiple views of the horizontal rod that can be used with the support arm system, for implementing an embodiment of the present disclosure.

FIGS. 13 and 14 provide multiple views of alternative clamp housings, for implementing embodiments of the present disclosure.

FIGS. 15A-C provide multiple views of a second alternative clamp housing, for implementing embodiments of the present disclosure.

FIG. 16 provides multiple views of a rounded mount for a pivoting rope cleat mount, for implementing embodiments of the present disclosure.

FIG. 17 provides multiple views of an alternative strut, for implementing embodiments of the present disclosure.

FIG. 18 provides a detailed isometric view of one embodiment of the alternative strut, for implementing embodiments of the present disclosure.

FIG. 19 provides multiple views of a single clamp housing having the capability of clamping two rods passing through corresponding substantially parallel channels in the clamp housing, for implementing embodiments of the present disclosure.

FIG. 20 provides multiple views of a single clamp housing having the capability of clamping two rods passing through corresponding, non-parallel channels in the clamp housing, for implementing embodiments of the present disclosure.

FIG. 21 is a flowchart diagram that illustrates the method operations performed in adjusting the support arm to a desired position, for implementing embodiments of the present disclosure

### DETAILED DESCRIPTION

Several exemplary embodiments for an adjustable support arm system, method and apparatus will now be described. It will be apparent to those skilled in the art that the present disclosure may be practiced without some or all of the specific details set forth herein.

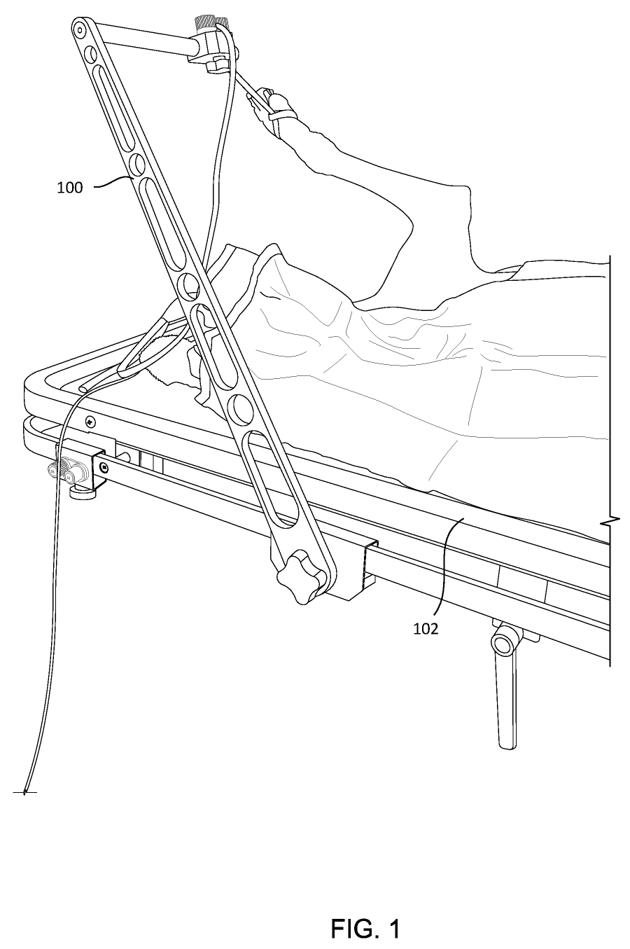

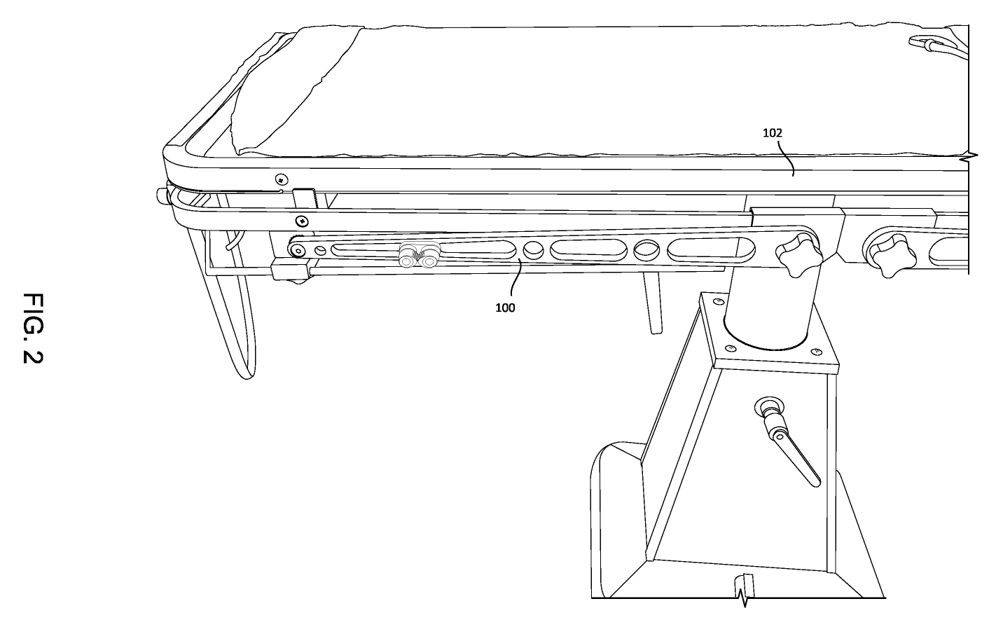

FIG. 1 is an illustration of the support arm 100 in use mounted on a worktable 102, for implementing embodiments of the present disclosure. In this implementation, the support arm 100 is used in a surgical environment to support the leg of a dog undergoing surgery. It should be understood that the support arm 100 could be used in any situation needing an easily adjustable support capable of being easily locked in many different positions.

FIG. 2 is another illustration of the support arm 100 in use, mounted on a worktable 102, for implementing embodiments of the present disclosure. In this instance, the support arm 100 is shown folded down in a substantially stowed configuration, while still being secured to the worktable 102. It should be understood that the support arm 100 can be raised, lowered and secured in a raised, lowered or any intermediate position between the raised and the lowered positions and can be moved side to side using a single control knob, as will be described in more detail below.

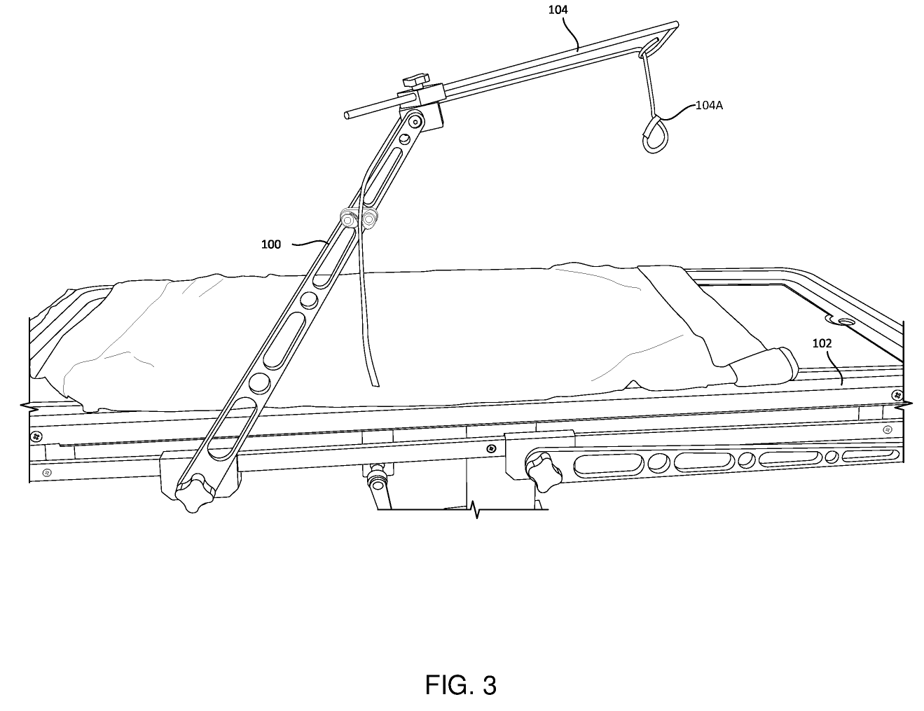

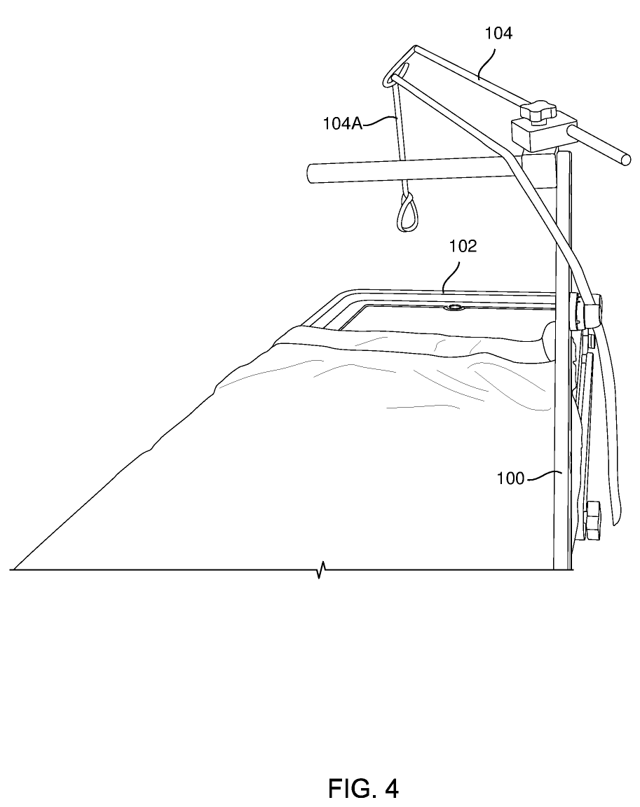

FIGS. 3 and 4 are additional illustrations of the support arm 100 in use mounted on the worktable 102, for implementing embodiments of the present disclosure. In this instance, the support arm 100 is shown using additional attachments to provide a sling support 104 that can be fixed in position as may be needed by the user. The sling support 104 can provide support for a sling 104A or other devices that may be supported by the sling support. Some additional attachments will be described in more detail below.

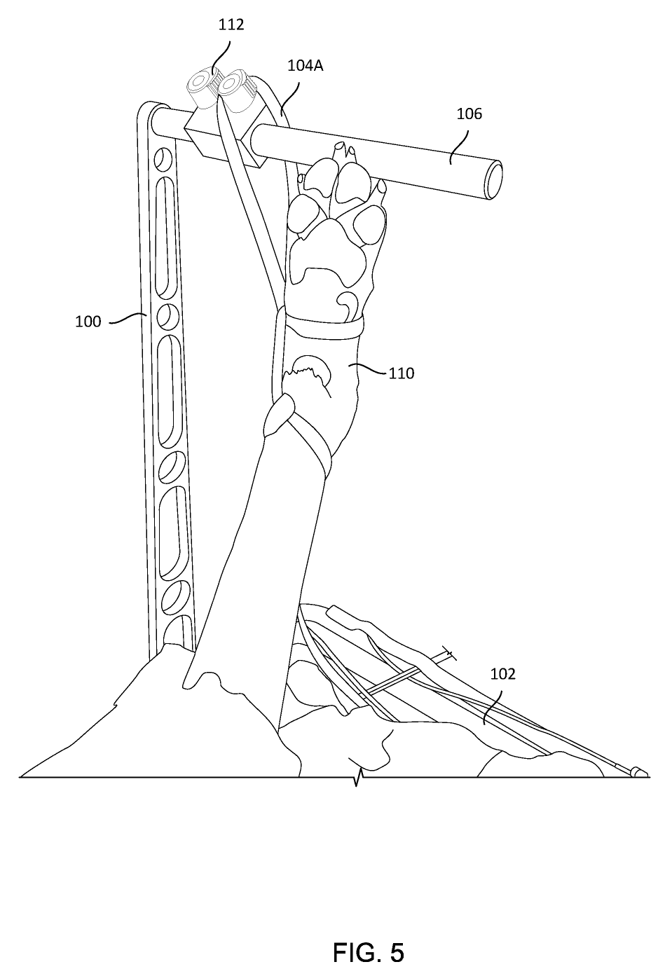

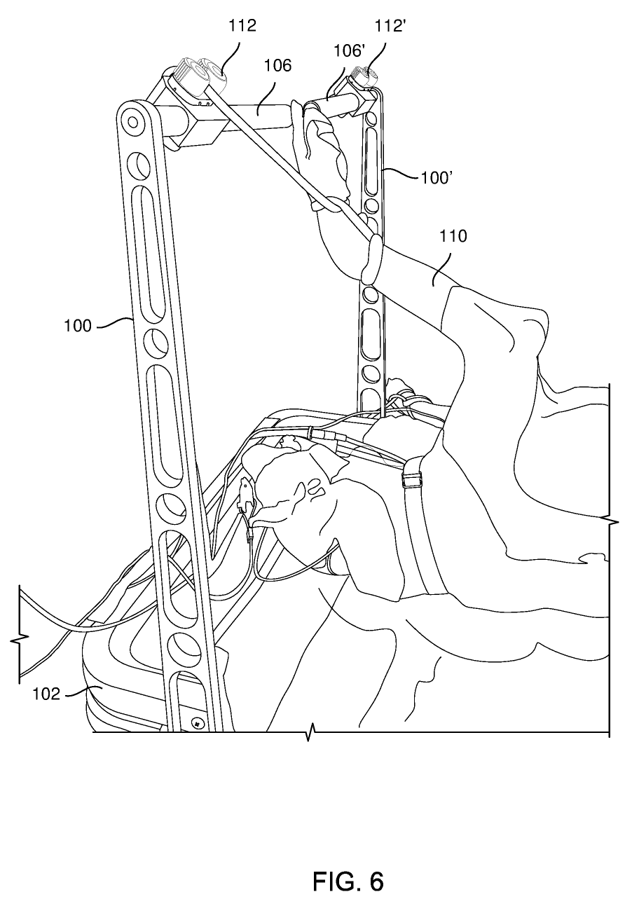

FIGS. 5 and 6 are additional illustrations of the support arm 100 supporting a horizontal rod 106 that can be used to support a workpiece 110, for implementing embodiments of the present disclosure. In this instance, the workpiece 110 is a veterinarian patient's leg. The workpiece 110 is secured in the desired position using a sling 104A that is held in place using a clamp 112 to allow the veterinarian to perform a medical procedure on the patient's leg. FIG. 6 further shows a second support arm 100′ secured to the worktable 102 with a second horizontal rod 106′ and a second clamp 112′ that may be used to support other portions of the patient or other instruments.

It should be understood that the support arm 100 can be used in many different configurations and implementations and in many different settings and is not limited to the medical field or even a veterinarian's field as a support arm could be used in a laboratory in a manufacturing environment, a mechanic's environment or many other situations where an adjustable support arm system that can be easily fixed in position would be useful. It should also be noted that the worktable 102 can be a workbench or other work area.

As shown in the above figures, the adjustable support arm system, method and apparatus can be implemented as a support structure capable of being secured to a work table or similar base structure. The support structure includes a base clamp capable of securely latching to the work table or similar base structure. The support structure also includes a primary arm having a base end coupled to the base clamp through a lockable, rotating base fastener. The primary arm further including a support end, the support end being an opposite end of the primary arm from the base end. The support end having a support post secured to the support end. The support post protruding from at least a first face of the primary arm. In at least one embodiment, the support post includes a substantially cylindrical shape, however it should be understood that the support post can have other shapes such as rectangular or triangular or other shape.

A single knob type lockable support clamp can be disposed on the support post. The single knob type lockable support clamp including a radiused locking face for engaging a corresponding radiused portion of the surface of the support post. The single knob type lockable support clamp also includes a single knob coupled to the radiused locking face. The single knob can selectively apply a clamping force, through the radiused locking face, to the corresponding radiused portion of the surface of the support post, when the single knob is turned in a locking direction. The single knob can also release the clamping force from the radiused locking face and the corresponding radiused portion of the surface of the support post, when the single knob is turned in an unlocking direction.

The support end of the primary arm can optionally and additionally include one or more detents disposed in the first face of the primary arm in a substantially circular pattern around the support post. The single knob type lockable support clamp can optionally also include one or more detent pins in a detent pin face of the single knob type lockable support clamp. The one or more detent pins can engage a corresponding one or more detents in the first face of the primary arm.

The support end of the primary arm can optionally and additionally include one or more detent pins disposed in the first face of the primary arm. The single knob type lockable support clamp can optionally also include a plurality of detents disposed in a detent face of the single knob type lockable support clamp in a substantially circular pattern around a support post channel in the single knob type lockable support clamp. The one or more detent pins can engage a corresponding one or more detents in the detent face of the single knob type lockable support clamp.

The single knob type lockable support clamp can optionally also include one or more springs for releasing the clamping force from the radiused locking face and the corresponding radiused portion of the surface of the support post, when the single knob is turned in an unlocking direction. The one or more springs can include a compressible solid spring material such as a rubber, silicon or similar material that will compress when the clamping force is applied and expand when the clamping force is released. The one or more springs can include a wavy-type spring washer that compresses to substantially flat when the clamping force is applied and will expand to the wavy shape as the clamping force is released. The one or more springs can include a spiral wound spring that compresses in length when the clamping force is applied and will rebound and extend when the clamping force is released. The one or more springs can include any suitable spring type material and configuration capable of storing compressed energy as the clamping force is applied and releasing the stored compressed energy when the clamping force is released thereby causing the radiused locking face to release the clamping force from the corresponding radiused portion of the surface of the support post.

The radiused locking face can be formed in a locking block that is coupled to the single knob. The locking block can optionally include an offset step to direct and increase the clamping force applied by the radiused locking face to the corresponding radiused portion of the surface of the support post when the single knob is turned in the locking direction.

The single knob type lockable support clamp can optionally also include one or more accessory mounts, IV clamps, secondary support posts, or other suitable accessory devices and supports. The single knob type lockable support clamp can optionally also include an indexed disk in a detent face in a second surface of the single knob type lockable support clamp. The single knob type lockable support clamp can optionally also include a duplex locking support clamp.

The support arm system components can be formed from any suitable materials including one or more and combinations of plastic, composites, ceramics, metals, aluminum, aluminum alloys, steel, steel alloys, and combinations thereof and any suitable materials having the physical qualities for the intended application such as strength, rigidity, electrical conductance, cleanliness, surface finish quality, capable of being sterilized.

Surface finish can be one or more of textured, knurled, polished, electropolished, anodized, plated, or other material finish suitable for the intended purpose. Physical sizes such as the lengths, thicknesses and widths depend on the desired use and the construction materials selected.

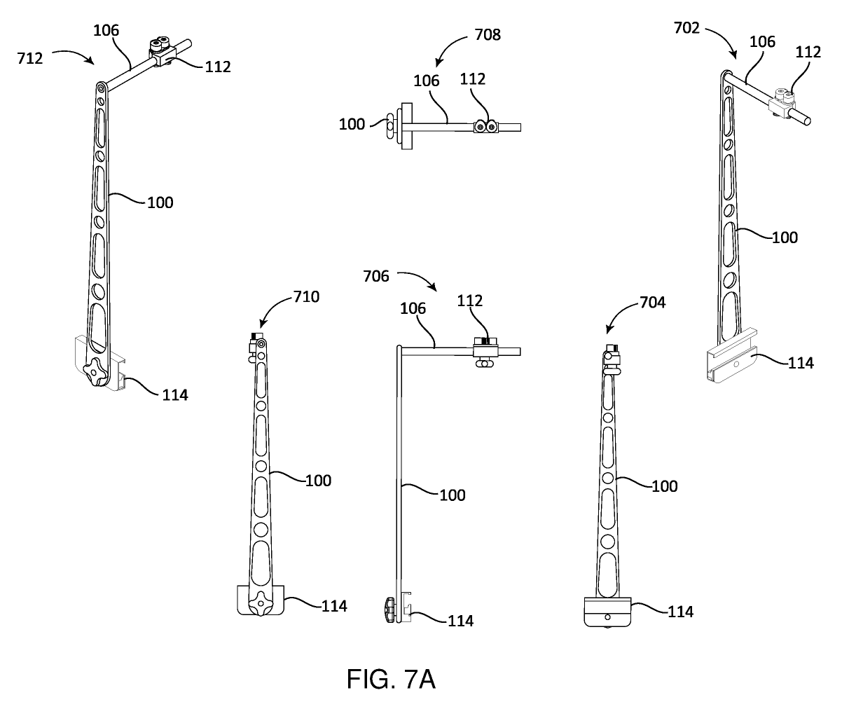

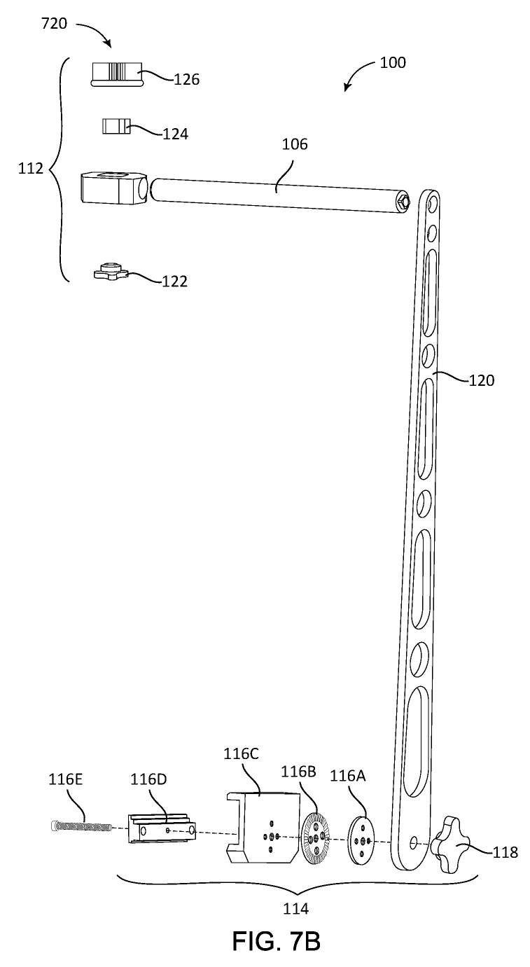

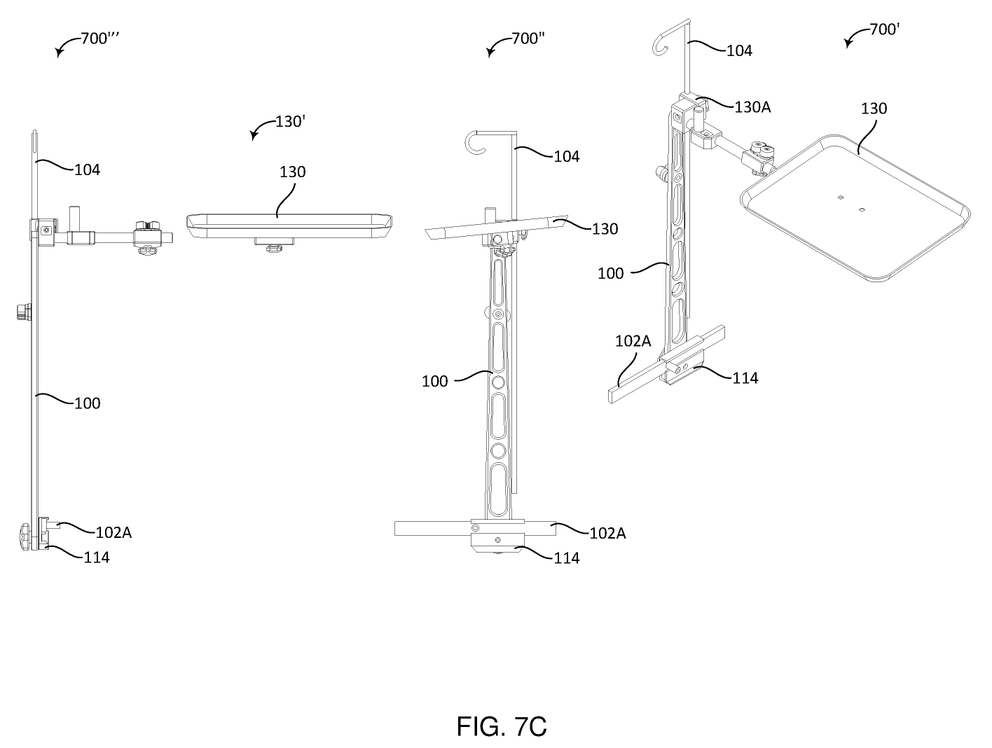

FIG. 7A is a simplified schematic diagram of several views 702-712 of the support arm 100, for implementing embodiments of the present disclosure. The support arm 100 is coupled to the horizontal rod 106 and a clamp 112 on the horizontal rod in one relatively simple implementation of the support arm. Additional implementations of the support arm and additional details of the individual elements of the support arm are described in the drawings herein.

FIG. 7B is a schematic diagram of an exploded view of one implementation of the support arm 100, for implementing embodiments of the present disclosure. The support arm 100 includes a base clamp assembly 114 for securing the support arm to the worktable (not shown), a friction system 116A, 116B for providing and controlling the friction in the up and down rotation of the support arm, a control knob 118 for controlling the friction system, a strut 120 forming the length of the support arm, the horizontal rod 106 and one example of a clamp 112 that can be secured to the horizontal rod. As will be described in more detail below the clamp can also include a control knob 122, a second friction system 124 and a latching system 126.

FIG. 7C is another schematic diagram of another use of the support arm 100, for implementing embodiments of the present disclosure. FIG. 7C includes an isometric view 700′, a front view 700″, a side view 700′″ of the support arm 100 supporting a pan 130. Also included is a side view 130′ of the pan 130. In this implementation, the support arm 100 is supporting a pan 130 and a sling support 104. It should be noted that the pan 30 and the sling support 104 are secured to the support arm system using single knob control clamps as will be described in more detail. Also shown in FIG. 7C is the base clamp 114 clamped to a support portion 102A of the worktable 102 shown in FIGS. 1-6 above.

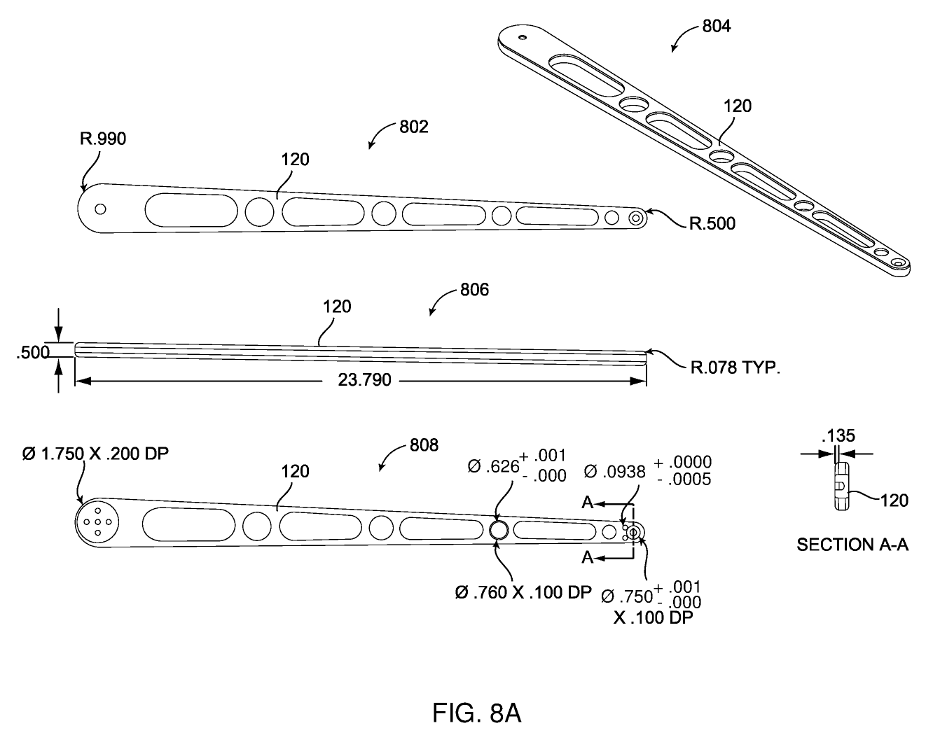

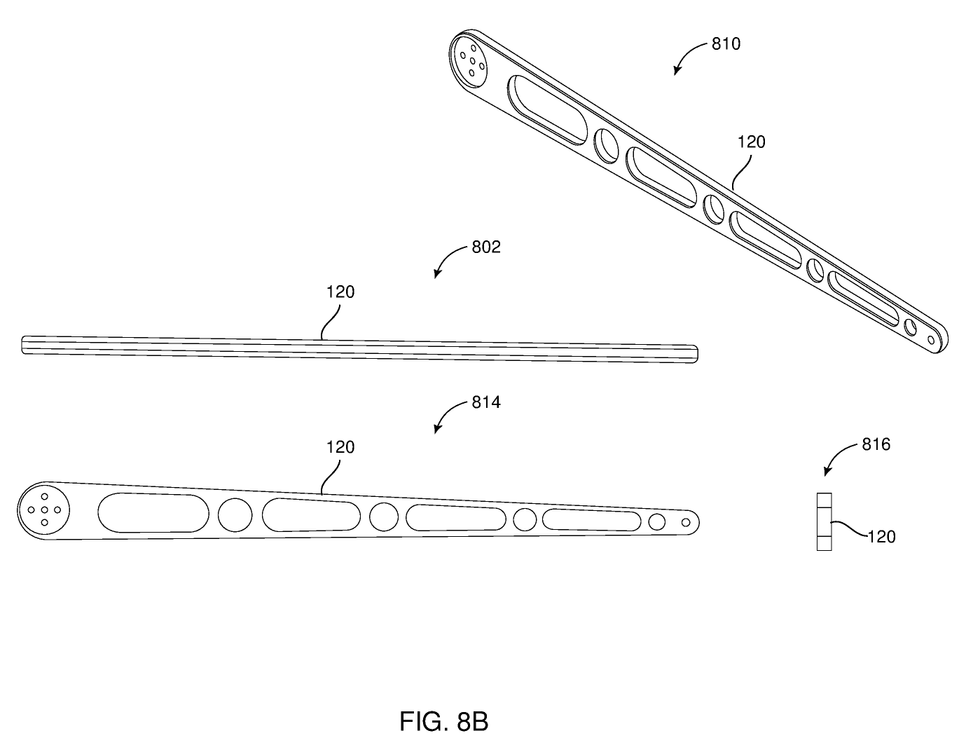

FIGS. 8A and 8B are exemplary detail views 802-816 of corresponding implementations of the strut 120 that could be used as part of the support arm system 100, for implementing embodiments of the present disclosure. It should be understood that the strut 120 can be formed of varying dimensions and varying materials as may be desired for the specific application. By way of example, the strut 120 can be formed from a metal such as stainless steel or aluminum or other suitable metal or combinations thereof. The strut 120 can be cast or machined or even stamped or forged or formed in an additive manufacturing process such as one or more 3D printing processes. In other implementations, the strut 120 may be formed of a plastic or ceramic or composite material such as a polyester resin or carbon fiber or fiberglass or any other suitable materials and combinations thereof. The specific dimensions of length and width of the strut 120 and whether or not there are lightening holes, as shown, or other decorative measures are selectable implementations that depend on the desired functional demands placed on the strut. Some exemplary dimensions are given for one implementation but should not be considered as the only possible imitation or limiting the possible implementations of the strut 120. The surface finishing can be any suitable finish as described elsewhere herein. Edges of the strut 120 or other structures and components described herein may be radiused or not radiused. Surfaces of the strut 120 or other structures and components described herein may be polished or not polished, plated or not plated, anodized or not anodized, painted or not painted, powder coated or not powder coated or any other suitable surface finish and combination of surface finishes.

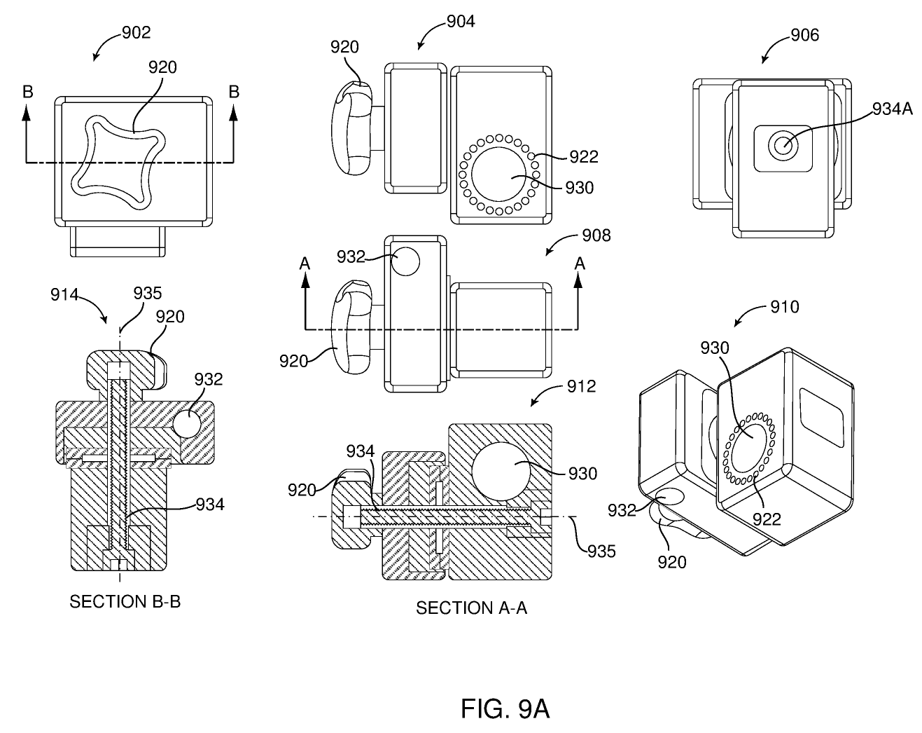

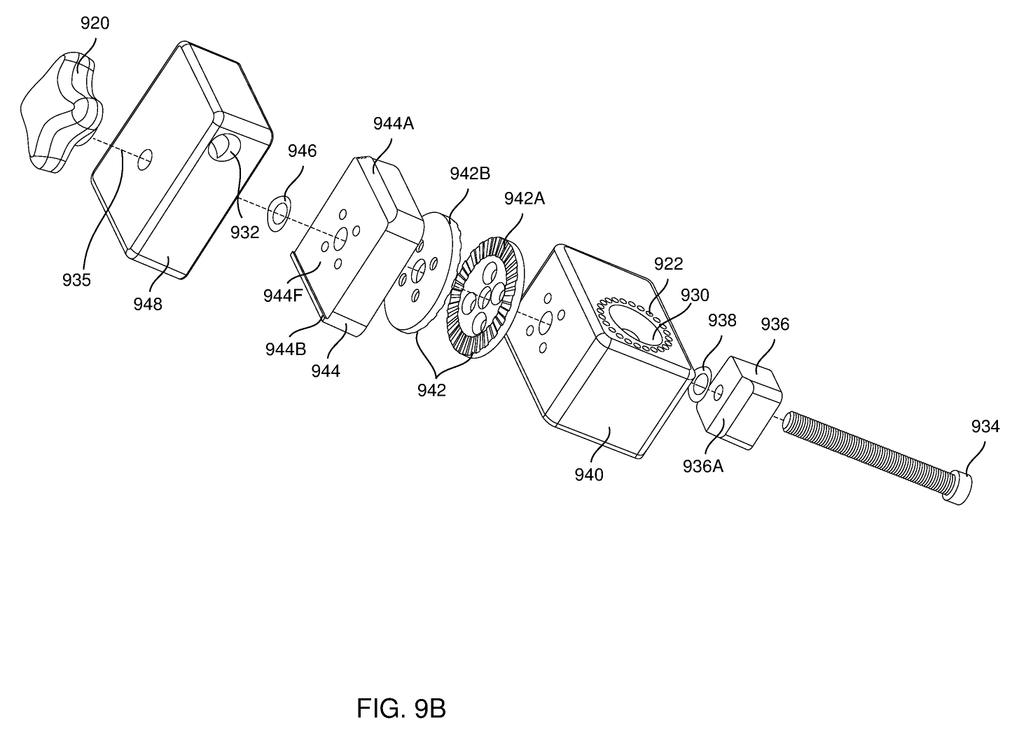

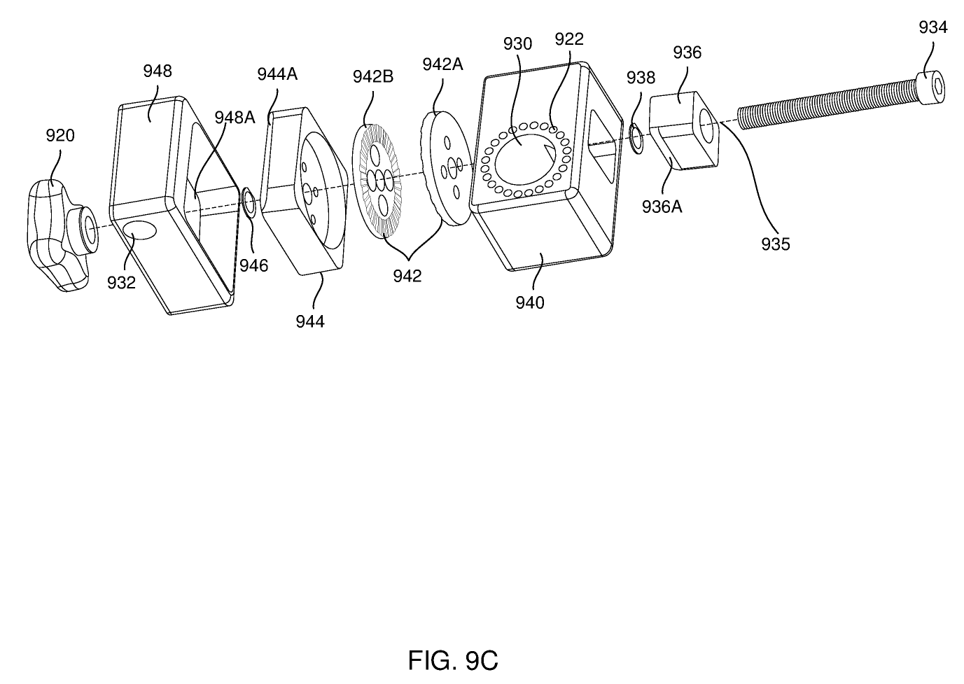

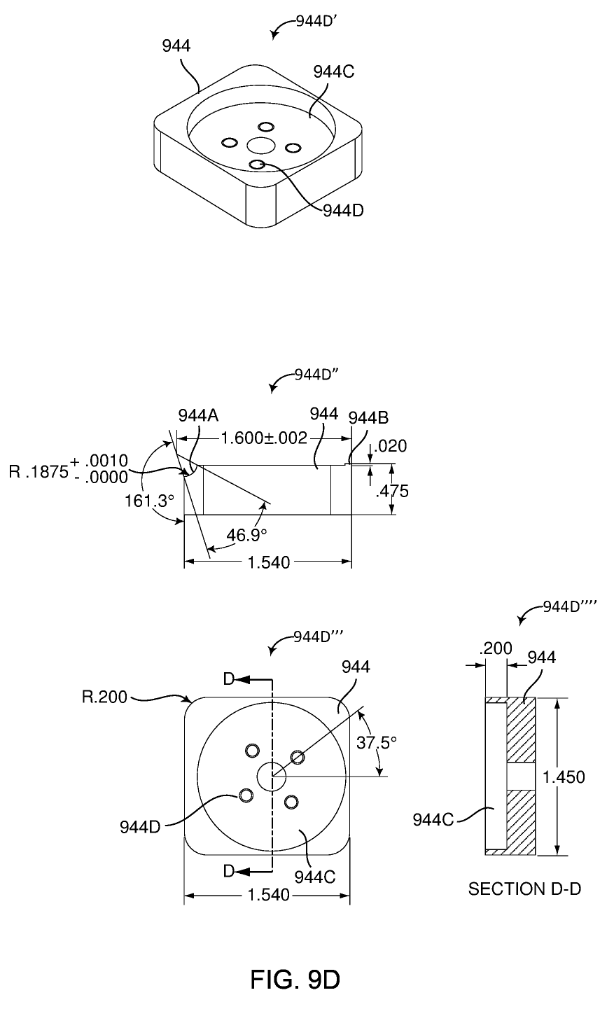

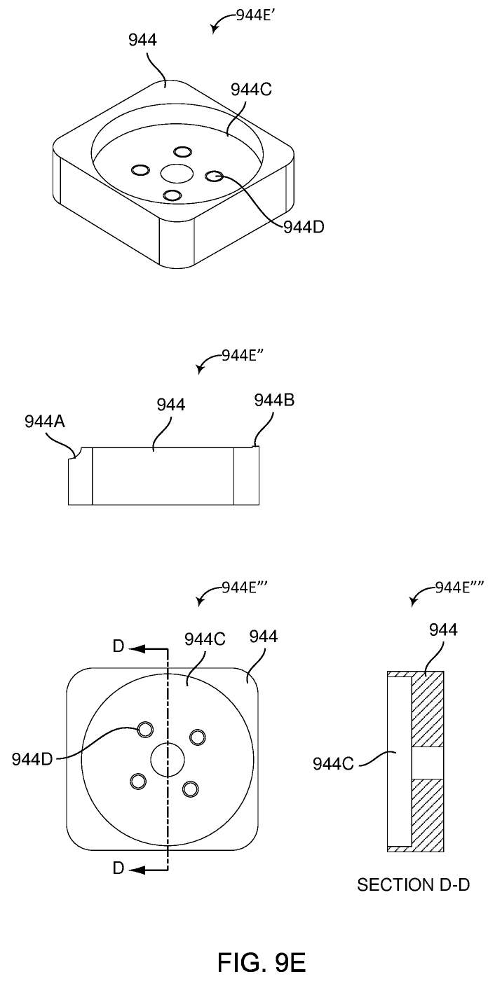

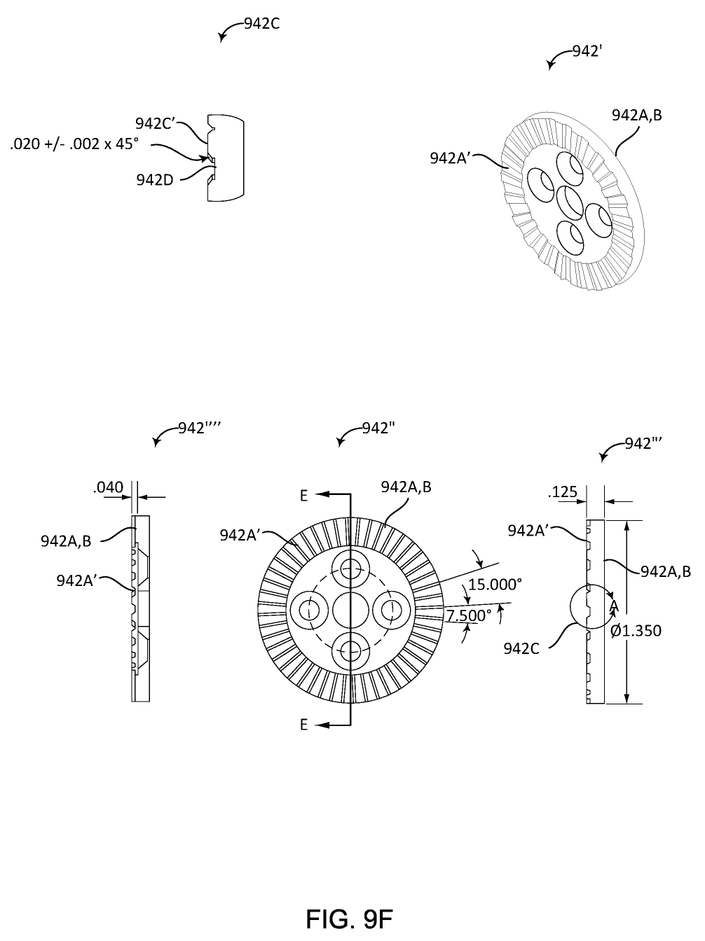

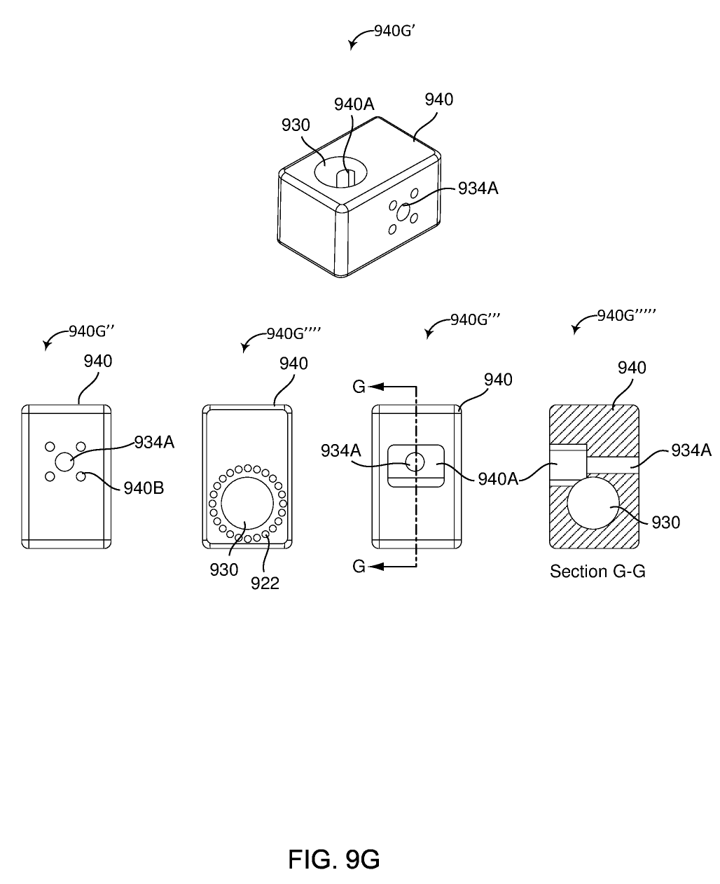

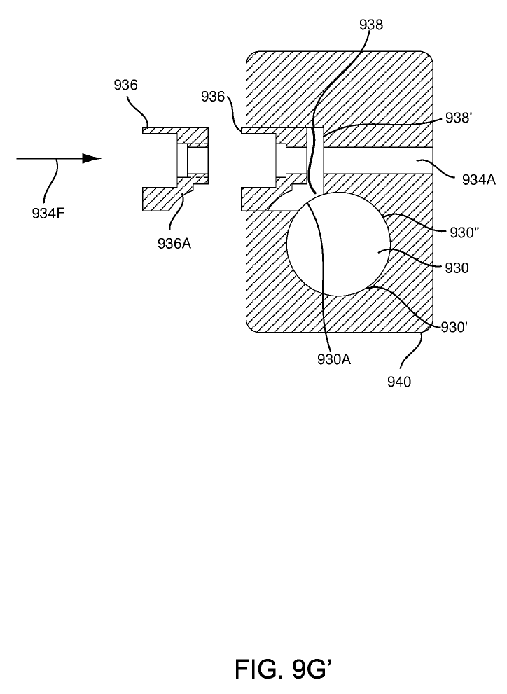

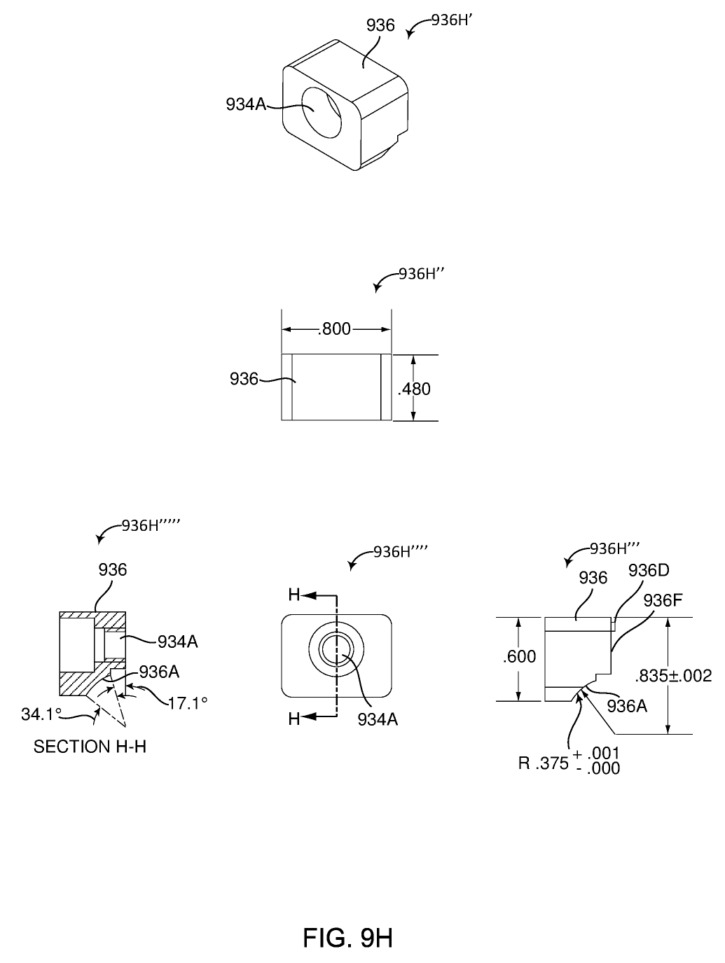

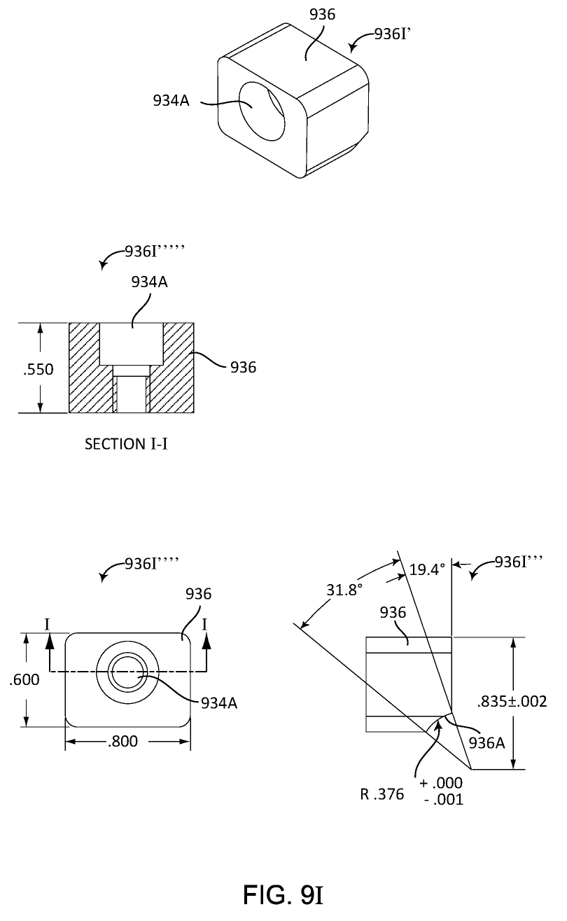

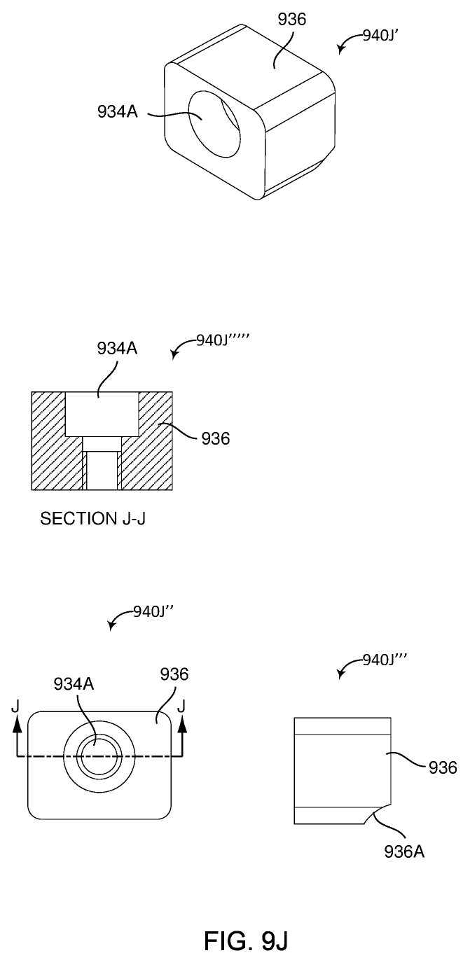

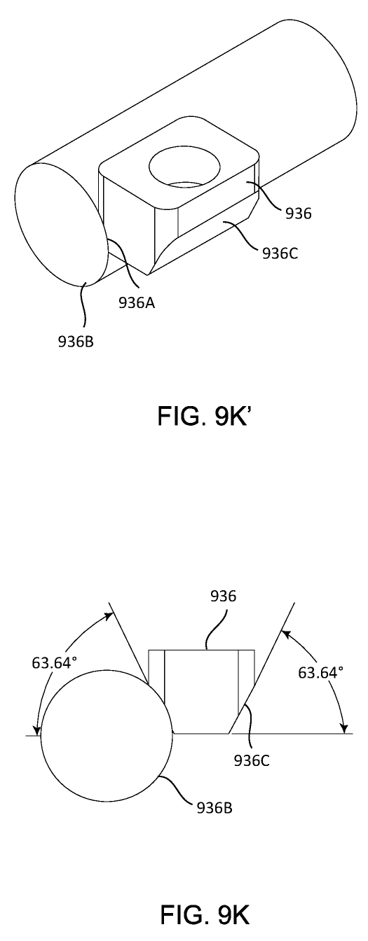

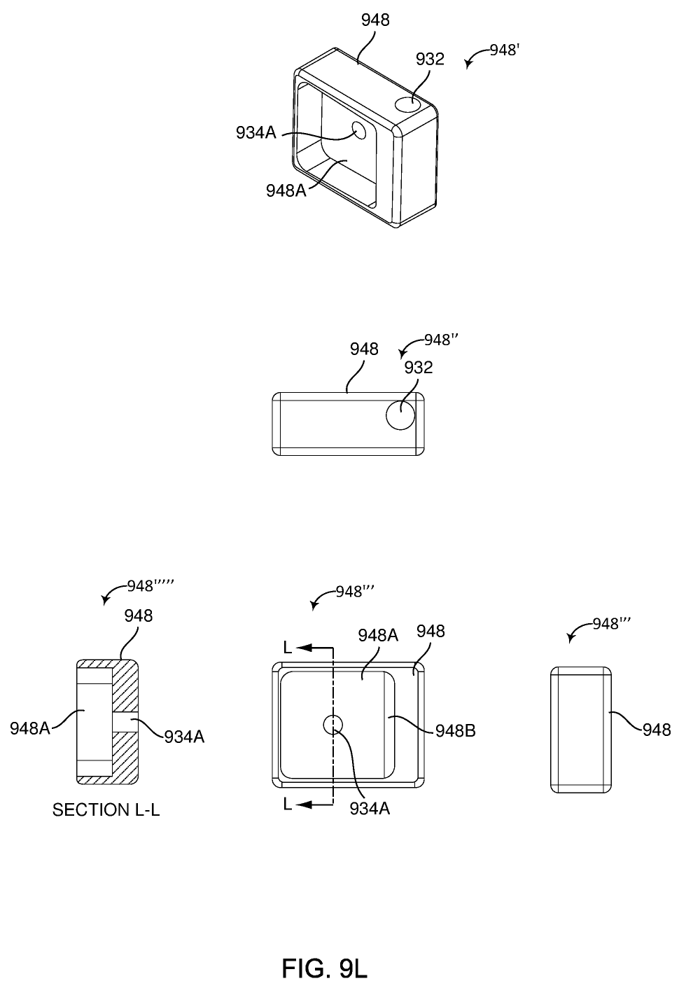

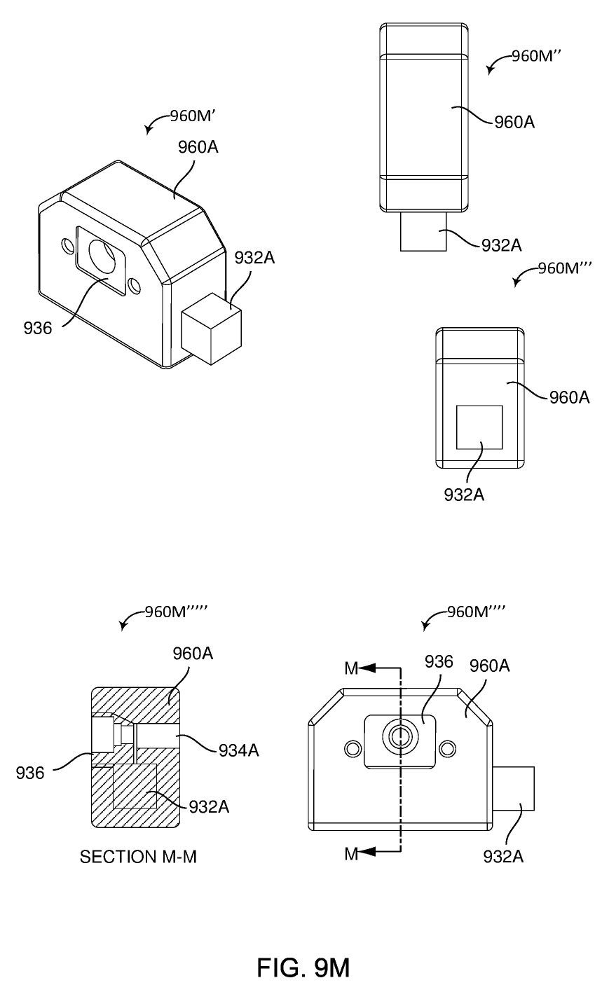

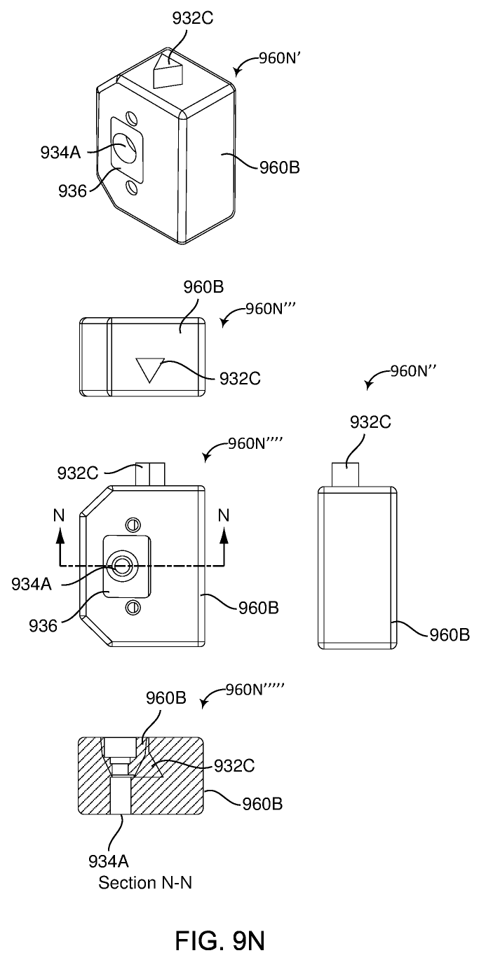

FIG. 9A provides multiple views 902-916 of a single control knob, multiple orientation, clamp system 900 that can be used with the support arm system or could be used in other implementations, independent of the support arm system, for implementing embodiments of the present disclosure. The single knob, multiple orientation, clamp system 900 provides a single control knob 920 that can control multiple planes of movement and rotation with the single control knob. In at least one implementation, the single control knob, multiple orientation, clamp system 900 includes multiple indexing holes 922 that can selectively engage one or more indexing pins that can lock portions of the single control knob, multiple orientation, clamp system. It should be understood that the indexing holes 922 and indexing pins (not shown) that engage the indexing holes are optional implementations. The disclosed implementation in FIG. 9A allows one smaller diameter rod (not shown) to pass through a correspondingly smaller sized passage 932 through the single control knob, multiple orientation, clamp system 900. A larger diameter rod, such as the horizontal rod 106, described above, can pass through a corresponding passage 930 through the single control knob, multiple orientation, clamp system 900. Having both the smaller rod and the larger rod passing through the single control knob, multiple orientation, clamp system 900 allows both the larger diameter rod and the smaller diameter rod to be adjusted in their respective orientations and also sliding along the length of the respective rods, all controlled by the single control knob 920. Cross-sectional views A-A and B-B are provided for more detailed views.

FIGS. 9B and 9C are exploded views 916, 918 of the single knob, multiple orientation, clamp system 900, for implementing embodiments of the present disclosure. The single control knob 920 is on the left and aligns with the control screw 934 on the right. The control screw 934 passes from the right, through a first cleat block 936, then a first wavy washer spring 938, then into a first clamp housing 940, then through a friction system 942 of two indexed discs 942A, 942B, then through a second cleat block 944, then a second wavy washer spring 946, then a second clamp housing 948 and ultimately to the single control knob.

In operation, the first clamp housing 940 and the second clamp housing 948 can rotate around the centerline 935. As the single control knob 920 tightens on the control screw 934, the first clamp housing 940 and the second clamp housing 948 are drawn together with increasing tension, thus compressing the springs 938 and 946 and the friction system 942 thus increasing friction between the first clamp housing and the second clamp housing and limiting a freedom of rotation around the centerline 935 between the clamp housings.

While not shown in this drawing, a smaller diameter rod can pass through the smaller diameter rounded channel 932 through the second clamp housing 948, and a larger diameter rod can pass through the larger diameter rounded channel 930 through the first clamp housing 940. Further rotating the single control knob 920 provides further tension on the control screw 934 which compresses the first spring 938 and second spring 946 and presses respective rounded surfaces 936A, 944A of the first and second cleat blocks 936, 944 into the rounded channels passing through the respective first and second clamp housings thus providing a locking force to the respective larger diameter rod and smaller diameter rod with the single control knob. It should be understood that other types of springs, as are described elsewhere herein, or no springs, could be used, instead of the wavy washer style springs 938, 948, shown in FIGS. 9B and 9C.

FIGS. 9D and 9E are multiple views of the second cleat block 944, for implementing embodiments of the present disclosure. The multiple views include isometric views 944D′, 944E′, top views 944D″, 944E″, front views 944D′″, 944E′″ and cross-sectional views 944D″″, 944E″″ of the second cleat block 944. Exemplary, nonlimiting dimensions are given for reference purposes only and the dimensions may be adjusted to accommodate the desired use and functionality. In at least one implementation, a raised portion 944B on a bottom surface 944F of the cleat block 944, as shown in FIG. 9D, having an exemplary dimension of 0.020 inches (0.5 mm) is an optional implementation to direct and increase a clamping force applied by the rounded clamping surface 944A of the second cleat block. The raised portion 944B causes the cleat block 944 to tip slightly toward the clamping surface 944A as the raised portion 944B contacts the bottom surface 948A of the cleat channel 948. Tipping the cleat block 944 toward the clamping surface 944A directs more of the clamping force toward the rod passing through the rod channel 932. Four optional holes 944D are shown for securing a portion 942B of the friction system 942 to the second cleat block 944. In the disclosed implementation, the friction system 942 is shown in an optional cavity 944C formed within the second cleat block 944. In alternative implementations, the optional cavity 944C could be less deep or even non-existent. Cross-sectional views section D-D are shown to illustrate the depth of the optional cavity 944C.

FIG. 9F are multiple views of the friction system 942, for implementing embodiments of the present disclosure. The multiple views include isometric view 942′, front view 942″, side view 942′″ and cross-sectional view E-E 942″ and detail view 942C of the indexed discs 942A, 942B. In the disclosed embodiment, the friction system 942 includes at least two substantially interlocking, indexed discs 942A, 942B. Detailed view 942C provides a more detailed view of raised portions 942C′ of the indexed surface 942A′ alternating with substantially equally spaced lowered portions 942D. A cross-sectional view E-E is provided to show detail of the indexing. A detailed view 942C of a portion of the cross-sectional view E-E is also provided.

In operation of at least one implementation, the indexed discs 942A, 942B are oriented so that the indexed surfaces 942A′ of each of the indexed discs are in contact, as described above, in FIGS. 9B and 9C, such that each raised portion 942C′ can intermesh with and extend into a corresponding lowered portion 942D. In this instance, the indexing of the raised portions 942C′ and lowered portions 942D is every 7½° around the circumference of the indexed discs, however it should be understood that it could be as few as two or three indexes in a full rotation of the indexed discs or as fine as more than one hundred indexes in the full rotation of the indexed discs. In another implementation of the friction system 942 not shown, the friction system can include one or more friction materials such as a rubber or plastic material or simply a roughened surface on the desired component that when pressed together increase the resistance i.e. friction thus limiting the freedom of movement between the two surfaces 942A′, in a substantially infinite number of increments of rotation.

FIG. 9G provides multiple views of the first clamp housing 940, for implementing embodiments of the present disclosure. An isometric view 940G′, a top view 940G″, a front view 940G′″, a side view 940G″″ and a cross-sectional view G-G 940G″″ of the first clamp housing 940 are provided. The rounded channel 930 allows a rounded rod to pass through the first clamp housing 940. A cleat channel 940A is provided for a cleat block 936 (not shown) to pass into. Note the cleat channel 940A opens to a portion of the rounded channel 930. A control screw channel 934A passes through the first clamp housing 940 and the cleat block 936 so that the control screw 934 can pass through both the first clamp housing and the cleat block. Four optional holes 940B are shown for securing a portion 942A of the friction system 942 to the first cleat block 940.

In at least one implementation, the first clamp housing 940 also includes multiple indexing holes 922 that can engage one or more corresponding indexing pins. Alternatively, the indexing pins can be secured in one or more of the indexing holes 922 of the first clamp housing 940. Indexing pins can then align and couple with a corresponding indexing hole in an adjacent surface as will be described in more detail below, and thus limit the rotation of the first clamp housing with respect to the adjacent surface.

FIG. 9G′ provides a progressive, cross-sectional view G-G of the cleat block 936 moving into the cleat channel 940A of the first clamp housing 940, for implementing embodiments of the present disclosure. The cleat block 936′ is shown fully outside and aligned with the cleat channel 940A. The cleat block 936″ is also shown partially inside the cleat channel 940A and nearing the rounded channel 930.

The rounded clamping surface 936A engages a rounded rod passing through the rounded channel 930 as the control screw 934 pushes the cleat block 936 into the cleat channel 940A, compressing the spring 938 between the cleat block and a bottom surface of the cleat channel 938′ and into a surface of the rounded rod present in the rounded channel with an increasing clamping force 934F, in the direction shown, toward a bottom surface 938′ of the cleat channel. Pushing or pressing the cleat block 936 into a first portion 930A of the surface of the rounded rod in the rounded channel 930 with increasing clamping force 934F increases friction between the surface of the rounded rod and the rounded clamping surface 936A of the cleat block 936 and also pushes the rounded rod into opposing portions 930′, 930″ of the inner surface of the rounded channel 930. Sufficient force applied to the cleat block 936 increases the friction between the rounded clamping surface 936A and the round rod and between the rounded rod and the inner surfaces of the rounded channel to substantially prevent the first clamp housing 940 from rotating around the rounded rod. The increased force applied to the cleat block 936 also increases the friction between the rounded clamping surface 936A and the round rod and between the round rod and the portions of the inner surfaces 930′, 930″ of the rounded channel to substantially prevent the first clamp housing 940 from moving lengthwise along the rounded rod. In this manner, the single control screw can control the friction of rotation and the friction of lengthwise movement between the first clamp housing 940 and the rounded rod passing through the rounded channel 930.

The control screw channel 934A is shown substantially perpendicular to the rounded channel. In one or more other implementations, the control screw channel 934A may or may not be substantially perpendicular to the rounded channel 930.

FIGS. 9H-J provide multiple views 936H-J of the first cleat block 936, for implementing embodiments of the present disclosure. In each of FIGS. 9H-J, corresponding views include isometric views 936H′, 936I′, 936J′, side views 936H″″, 936I′″, 936J′″, front views 936H″″, 936I″″, 936J″″ and cross-sectional views 936H″″′, 936I″″′, 936J″″′. A top view 936H″ is provided in FIG. 9H. The first cleat block 936 includes the rounded clamping surface 936A that corresponds to the rounded surface of a rounded rod passing through the round channel 930 in the first clamp housing 940. Corresponding sectional views H-H, I-I and J-J are also provided.

The first cleat block 936 has an exterior perimeter shape to fit into a corresponding shaped cleat channel 940A in the first clamp housing 940. While the exterior perimeter shape of the first cleat block 936 and the corresponding shaped cleat channel 940A is shown as substantially rectangular, in other implementations, the exterior perimeter shape of the first cleat block and the corresponding shaped cleat channel can be square, round, triangular or any other suitable shape. The exterior perimeter shape of the first cleat block 936 and the corresponding shaped cleat channel 940A can also optionally include one or more indexing portions, e.g., intermeshing shapes, not shown, to maintain alignment of the first cleat block in the cleat channel.

In one implementation, the rounded clamping surface 936A of the first cleat block 936 is substantially radially aligned with a center of the round channel 930 in the first clamp housing. The radial length of the rounded clamping surface 936A of the first cleat block can be any suitable range as shown in one implementation the rounded clamping surface is 34.1 degrees in another implementation the rounded clamping surface is 31.8 degrees. However, it should be understood that these are just exemplary embodiments and that the radial length of the rounded clamping surface 936A of the first cleat block 936 can be within a range of about 20 degrees to about 90 degrees. In at least one embodiment, the rounded clamping surface 936A of the cleat block 936 substantially matches a rounded surface shape of the rounded rod passing through the rounded channel 930. In at least one alternative, the rounded clamping surface 936A of the cleat block 936 can substantially not match the rounded surface shape of the rounded rod passing through the rounded channel 930. By way of example, the rounded clamping surface 936A of the cleat block 936 can be substantially flat or wedge shape and not rounded such that increased clamping force 934F drives the flat or wedge shape of the clamping surface into the rounded rod, thus increasing the friction as described above. The cleat block 936 can also include a raised portion 936D on a bottom surface 936F. The raised portion 936D causes the cleat block 936 to tip slightly toward the clamping surface 936A as the raised portion 936D contacts the bottom surface 938′ of the cleat channel 940A. Tipping the cleat block 936 toward the clamping surface 936A directs more of the clamping force toward the rod passing through the rod channel 930.

FIGS. 9K and 9K′ illustrate the rounded clamping surface 936A of the cleat block 936 engaging a rounded surface of the rounded rod 936B, for implementing embodiments of the present disclosure. FIG. 9K provides an end view of the rounded rod 936B and the cleat block 936. FIG. 9K′ provides an isometric view of the rounded rod 936B and the cleat block 936. The rounded rod 936B can pass through the round channel 930 in the first clamp housing 940. It should be understood that the second cleat block could engage a second rounded rod that passes through the rounded passage 932 in the second clamp housing 948 in a substantially similar manner, even though the diameters of the respective rounded rods could be substantially different or substantially the same.

Also shown in FIG. 9K, is an optional, exemplary, back cut 936C of approximately 63.64 degrees. The back cut 936C is used as a wedge against a corresponding cut in the cleat channel 940A of the first clamp housing 940 so that the cleat block 936 is pushed in a substantially radial direction toward the center of the rounded rod 936B passing through the rounded channel 930. Due to the corresponding equivalent angles of the back cut 936C and the engagement angle as shown in FIG. 9K. However, it should be understood that the 63.64 degrees is merely an exemplary embodiment and that other ranges of degrees for the back cut 936C and engagement angles can be used. Further, the engagement angle does not have to be the same as the back cut angle. By way of example the back cut 936C angle can be between about 30 to about 90 degrees and the engagement angle can be between about 30 to about 90 degrees.

FIG. 9L provides multiple views 948′-948″″′ of the second clamp housing 948, for implementing embodiments of the present disclosure. An isometric view 948′ a top view 948″, side view 948′, front view 948″″ and cross-sectional view L-L 948″″′ of the second clamp housing 948A are provided. The second clamp housing 948 has a corresponding second cleat channel 948A and a control screw channel 934A, for the control screw 934, passing substantially perpendicular to the rounded channel 932 and a portion of the rounded channel passes through the cleat channel so that the rounded clamping surface 944A of the second cleat 944 can engage the rounded rod passing through the rounded channel. In operation, the second cleat 944 engages the rounded rod passing through the rounded channel of the second clamp housing 948 in substantially similar manner as described above with regard to the first clamp housing as the clamping force 934F presses the second cleat into the second cleat channel 948A to engage the clamping surface 948B to the surface of the rounded rod passing though the rounded channel 932. Further and as described above, with regard to the first clamp housing, the clamping surface 948B can be substantially similar or not similar in shape to the surface of the rounded rod passing though the rounded channel 932.

It should be understood that while the disclosed embodiments described rounded rods having a rounded cross-sectional shape passing through corresponding rounded channels, in the respective clamp housings, rods having other shapes than a round cross-section can also be used. FIG. 9M illustrates multiple views of a clamp housing 960A engaging a rod having a rectangular cross-sectional shape, for implementing an embodiment of the present disclosure. An isometric view 960M′, a top view 960M″, a side view 960M″′, a front view 960M″″ and a cross-sectional view M-M 960M″″′ of the clamp housing 960A are provided. A rectangular cross-section rod 932A passes through a corresponding rectangular cross-section channel in the clamp housing 960A.

FIG. 9N illustrates multiple views of a clamp housing 960B engaging a rod having a triangular cross-sectional shape, for implementing an embodiment of the present disclosure. An isometric view 960N′, a top view 960N″, a side view 960N″′, a front view 960N″″ and a cross-sectional view N-N 960N″″′ of the clamp housing 960B are provided. A triangular cross-section rod 932C passes through a corresponding triangular cross-section channel in the clamp housing 960B. Similarly, other shapes such as T cross-sections, X cross-sections, ovals, or other cross-sectional shaped rods and corresponding housings could be used.

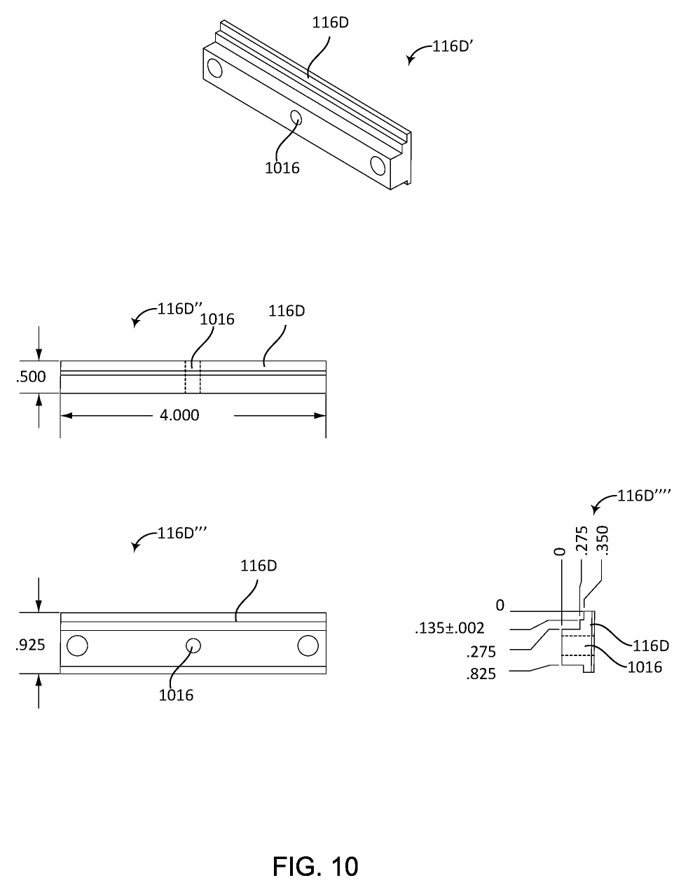

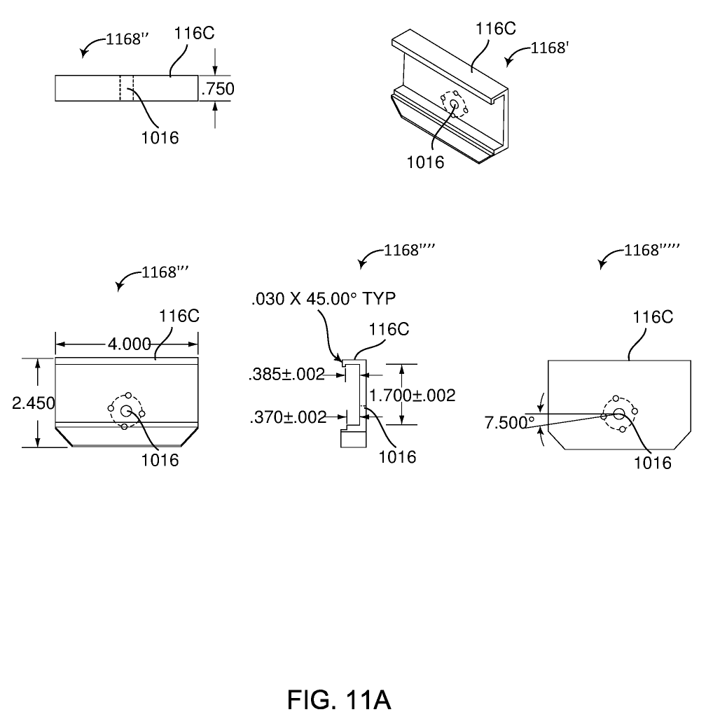

FIG. 10 provides multiple views of a clamp block 116D of the base clamp 114, for implementing an embodiment of the present disclosure. The multiple views include an isometric view 116D′, a top view 116D″, a front view 116D″′ and an end view 116D″″ of the base clamp block 116D. The base clamp block 116D clamps the sliding mount surface 102A on the worktable 102, as shown in the figures above.

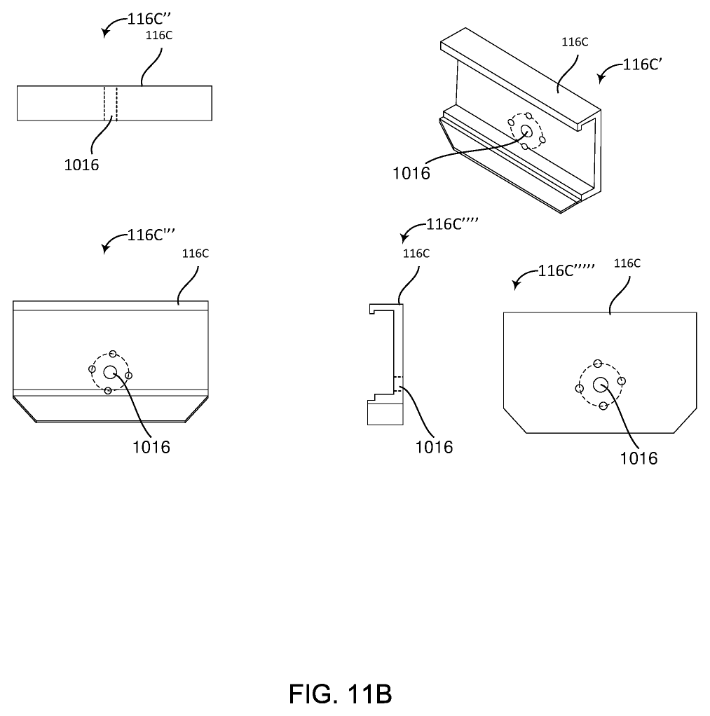

FIGS. 11A-11B provides multiple views of the base clamp housing 116C, for implementing an embodiment of the present disclosure. The multiple views include isometric views 116B′, 116C′, top views 116B″, 116C″, front views 116B″′, 116C″′, end views 116B″″, 116C″″ and rear views 116B″″′, 116C″″′ of the base clamp housing 116C. In operation, the base clamp housing is held on the outer surface of the sliding mount surface 102A of the worktable 102 and the clamp block 116D is held against the inner surface of the sliding mount surface. The base clamp housing 116C is held to the clamp block 116D by one of more clamping screws passing through one or more clamping screw channel 1016 and corresponding one or more clamping knob. When the one or more clamping knob is tightened, the base clamp housing is pulled toward the clamp block to clamp the sliding mount surface 102A of the worktable 102, between the base clamp housing and the clamp block, as shown in the figures above. The exemplary dimensions are given for example purposes only and it should be understood that the dimensions can vary depending on the specific application.

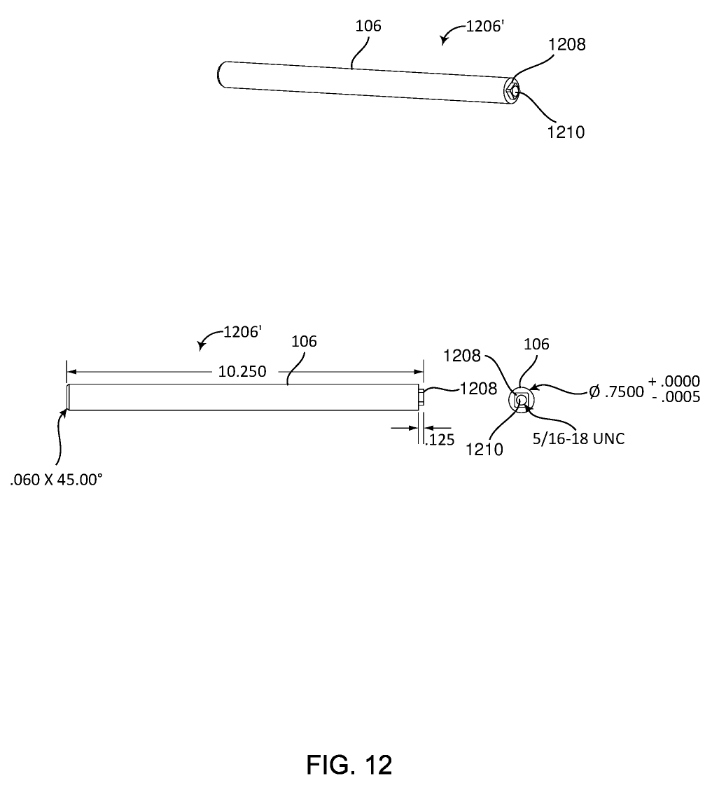

FIG. 12 provides multiple views of the horizontal rod 106 that can be used with the support arm system 100, for implementing an embodiment of the present disclosure. The multiple views include an isometric view 1206′, a side view 1206″ and an end view 1206″′ of the horizontal rod 106. As described above, the horizontal rod 106 may be a rounded cross-sectional shape or any suitable cross-sectional shape such as square, rectangle, triangular, oval, T, X, pentagonal, hexagonal, heptagonal, octagonal, or any other suitable shape. In at least one implementation, the horizontal rod 106 includes a keyed shape 1208 in at least one end. The keyed shape 1208 can be any suitable shape. The keyed shape 1208 shown is rectangular and extends from the end of the horizontal rod. The keyed shape 1208 can engage a correspondingly shaped intention or channel to in the strut 120 or a clamp housing so that the horizontal rod 106 does not rotate relative to the strut or clamp housing.

The horizontal rod 106 can also include a mounting hole 1210 for receiving a mounting hardware that can secure the horizontal rod to the strut 120 or a clamp housing. In at least one implementation, the mounting hole 1210 is threaded for receiving a threaded fastener such as a screw or a bolt, however, in other implementations, the horizontal rod can be secured to the strut 120 or clamp housing by any suitable means such as a rivet, welding, adhesive, or any other suitable means and combinations thereof. In at least one embodiment, the horizontal rod 106 can formed as part of the strut 120 or clamp housing.

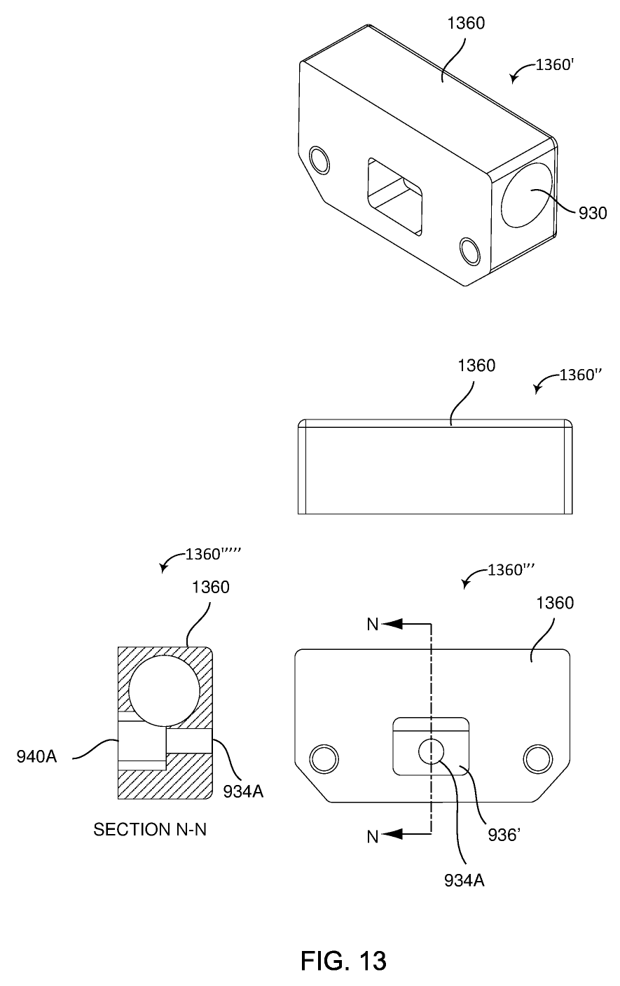

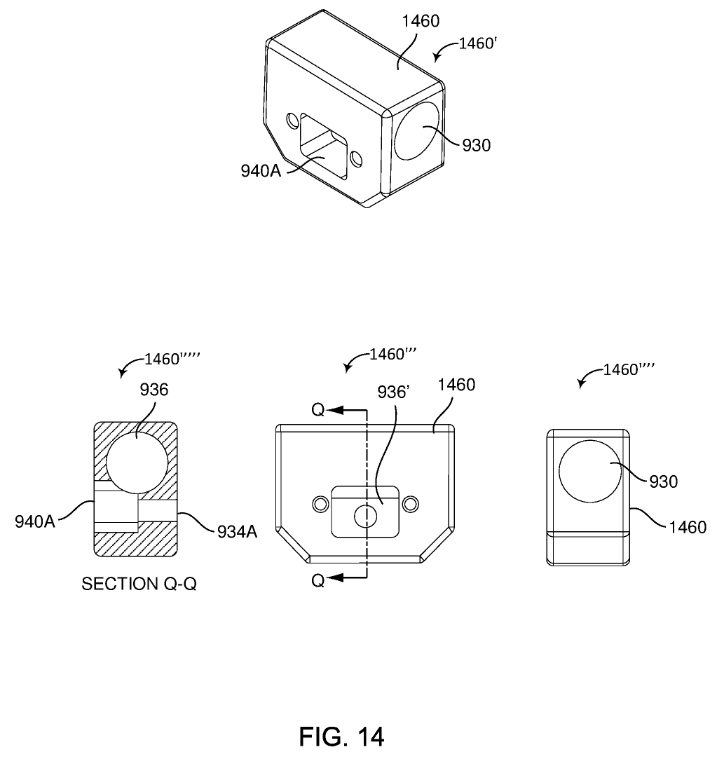

FIGS. 13 and 14 provide multiple views of alternative clamp housings 1360, 1460, for implementing embodiments of the present disclosure. The multiple views include isometric views 1360′, 1460′, a top view 1360″, front view 1360″′, 1460″′, side view 1460″″ and cross-sectional view N-N 1360″″′ and cross-sectional view Q-Q 1460″″′ of the alternative clamp housings 1360, 1460. The alternative clamp housings 1360, 1460 can use an alternative cleat block 936′ shape that operates similarly to the first and second clamp housings and cleat blocks 936, 944, described above. The alternative cleat block 936′ can have a clamping surface that includes a different radial length and/or orientation than the rounded clamping surface 936A of cleat blocks 936 or 944.

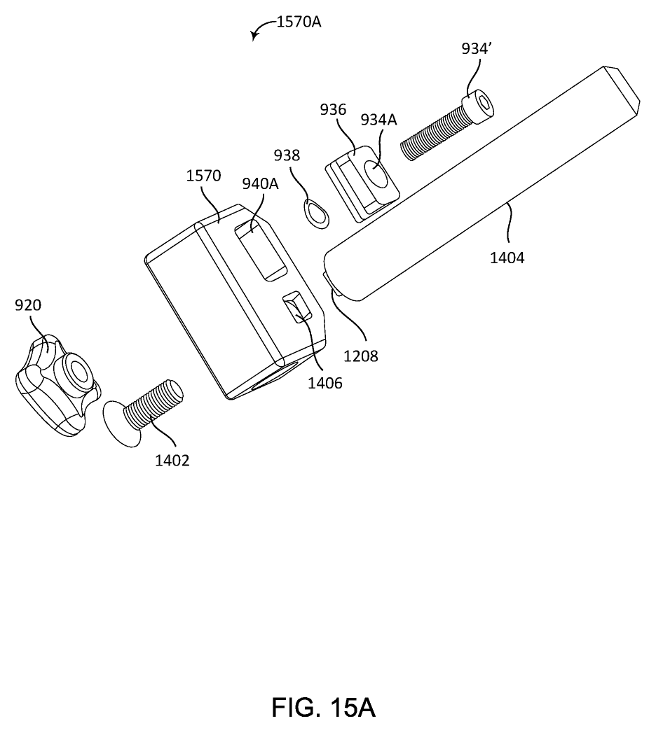

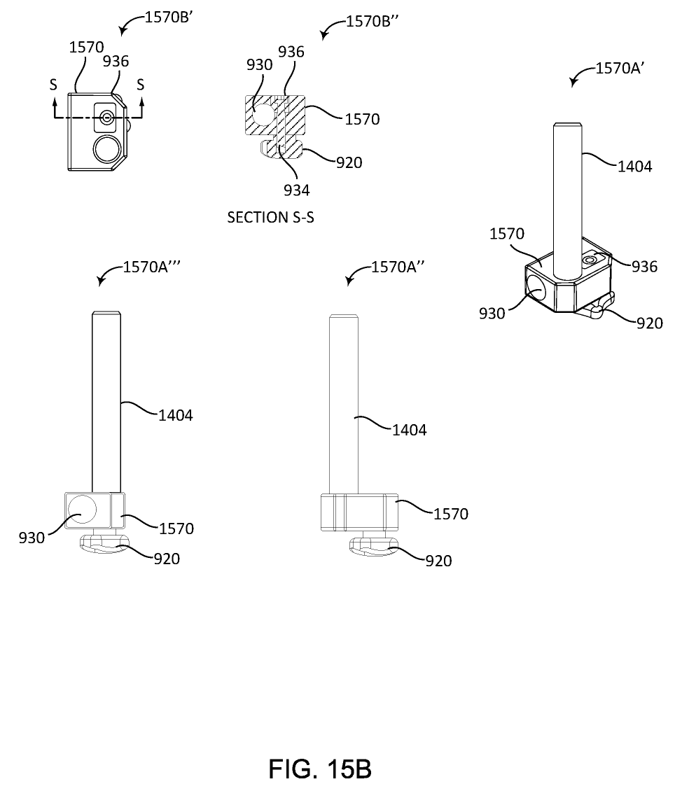

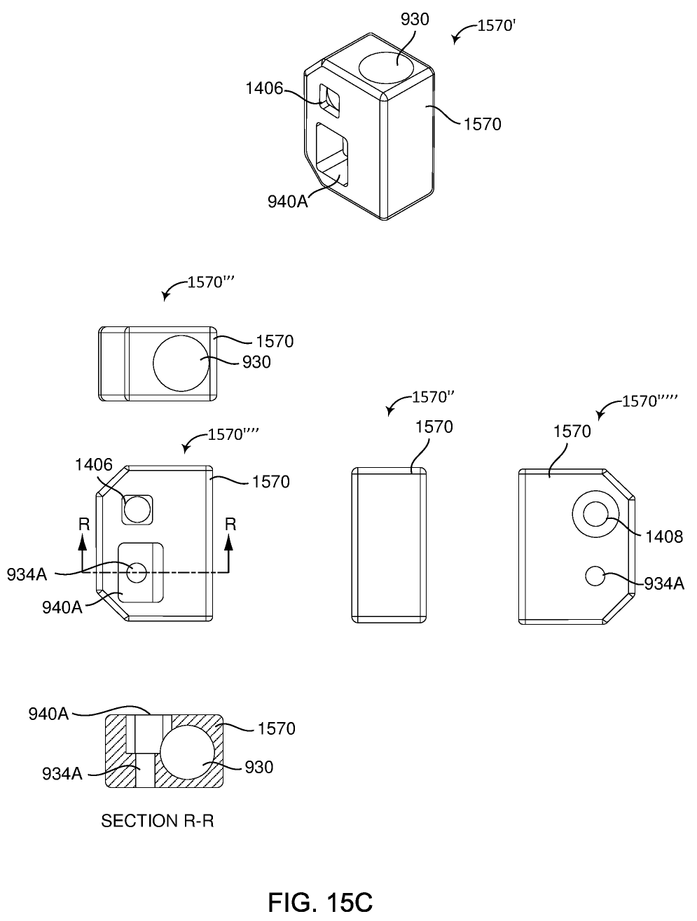

FIGS. 15A-C provide multiple views of a second alternative clamp housing 1570, for implementing embodiments of the present disclosure. FIG. 15A provides an exploded, isometric view 1570A of the second alternative clamp housing 1570 and an optional accessory bar 1404. FIG. 15B provides an isometric view 1570A′, a front view 1570A″, a side view 1570A″′ of the second alternative clamp housing 1570 and an optional accessory bar 1404. FIG. 15B also includes a front view 1570B′ and a cross-sectional view S-S 1570B″ of the second alternative clamp housing 1570. FIG. 15C provides an isometric view 1570′, a top view 1570″, an end view 1570″′, a front view 1570″″, a rear view″″′ and a cross-sectional view R-R 1570″″″ of the second alternative clamp housing 1570.

The second alternative clamp housing 1570 can include an optional accessory bar 1404. The optional accessory bar 1404 includes a keyed end 1404A having a keyed shape, in this instance a rectangular shape, that corresponds to a corresponding keyed cavity 1406 in the second alternative clamp housing 1570. A screw 1402 can thread into a correspondingly threaded opening in the end of the optional accessory bar 1404 to secure the optional accessory bar to the second alternative clamp housing.

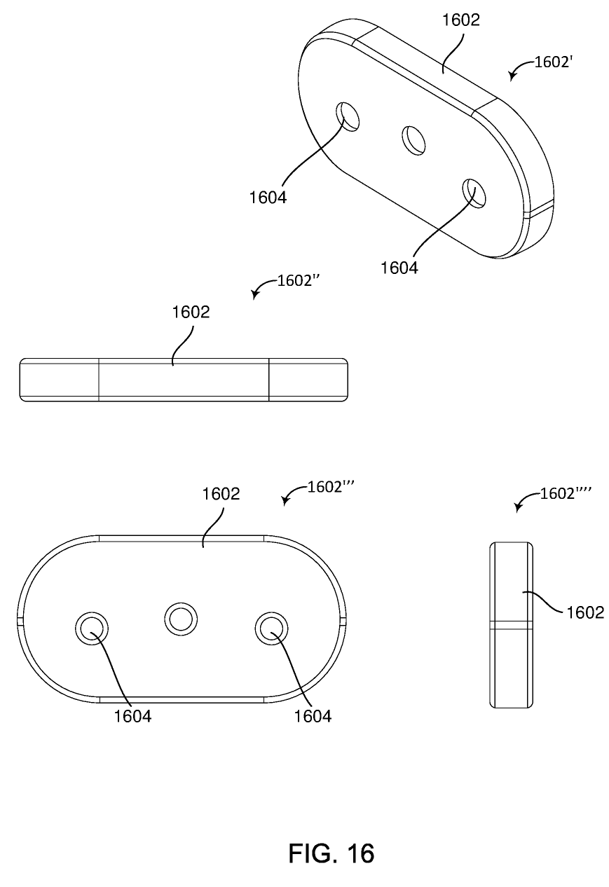

FIG. 16 provides multiple views of a rounded mount 1602 for a pivoting rope cleat mount, for implementing embodiments of the present disclosure. The multiple views include an isometric view 1602′, a top view 1602″, a front view 1602″′, and a side view 1602″″ of the rounded mount 1602. The rounded mount 1602 for the pivoting rope cleat mount can be mounted to any one of the clamp housings described herein. Two eccentric knobs having knurled surface texture as shown in FIGS. 1 and 2 can be mounted in the mounting holes 1604. In operation, the two eccentric knobs can rotate and clamp a rope or similar device as the rope is pulled between the two eccentric knobs.

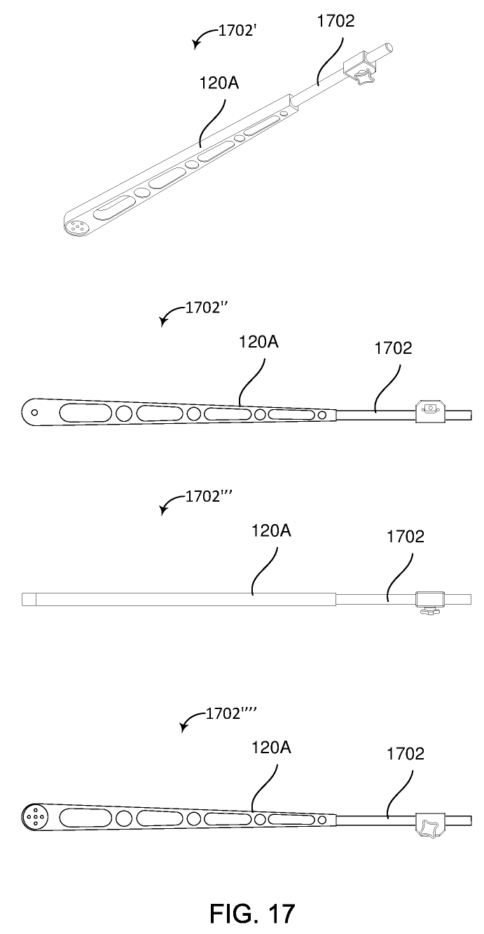

FIG. 17 provides multiple views of an alternative strut 120A, for implementing embodiments of the present disclosure. The multiple views include an isometric view 1702′, a top view 1702″, a side view 1702″′, and a rear view 1702″″. The alternative strut 120A includes a rounded mount rod 1702 coupled or formed substantially in line with the alternative strut.

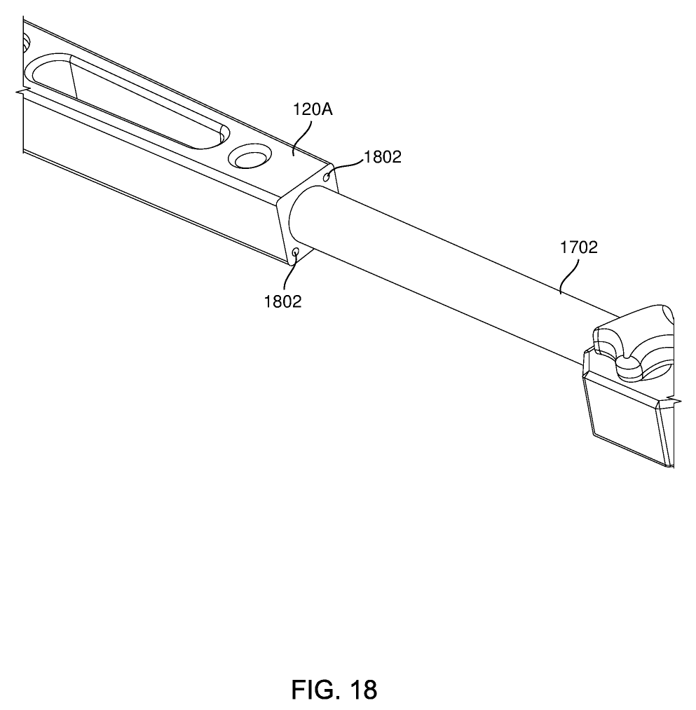

FIG. 18 provides a detailed isometric view of one embodiment of the alternative strut 120A, for implementing embodiments of the present disclosure. The alternative strut 120A includes one or more index holes 1802 and/or index pins for engaging a clamp mounted on the rounded mount rod 1702.

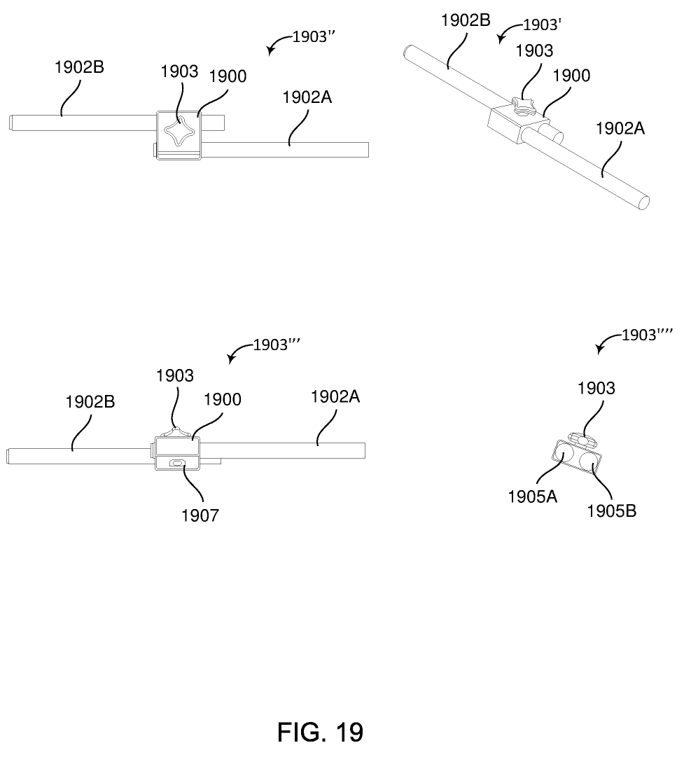

FIG. 19 provides multiple views of a single clamp housing 1900 having the capability of clamping two rods 1902A, 1902B passing through corresponding substantially parallel rod channels 1905A, 1905B in the clamp housing, for implementing embodiments of the present disclosure. The multiple views include an isometric view 1903′, a top view 1903″, a side view 1903″′ and an end view 1903″″ of the single clamp housing 1900. As shown in FIG. 19, the two rods 1902A, 1902B are substantially parallel and the two rods can be clamped in the corresponding rod channels 1905A, 1905B with a single control knob 1903 acting on one or more corresponding cleat blocks 1907 that are drawn into contact and provide clamping pressure to both rods 1902A, 1902B as the single control knob is tightened. The rod channels 1905A, 1905B can be the equal width or non-equal widths.

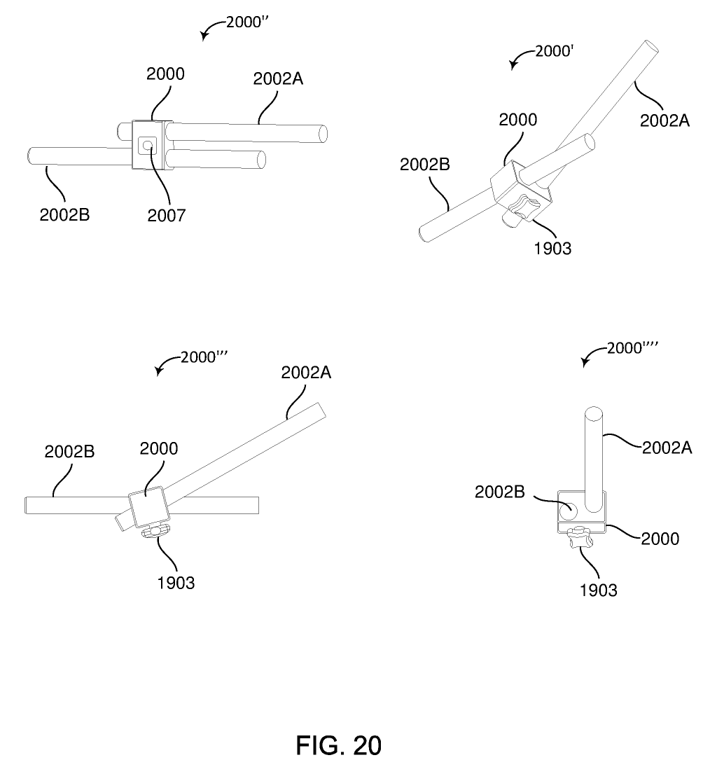

FIG. 20 provides multiple views of a single clamp housing 2000 having the capability of clamping two rods 2002A, 2002B passing through corresponding, non-parallel channels 2005A, 2005B in the clamp housing, for implementing embodiments of the present disclosure. The multiple views include an isometric view 2000′, a top view 2000″, a front view 2000″′ and an end view 2000″″ of the single clamp housing 2000.

The two rods 1902A, 1902B′ are substantially non-parallel and yet the two rods can be clamped with a single control knob 1903 acting on corresponding one or more cleat block(s) 2007 in the single clamp housing 2000 that are drawn into contact and provide clamping pressure to both rods 2002A, 2002B as the single control knob 1903 is tightened.

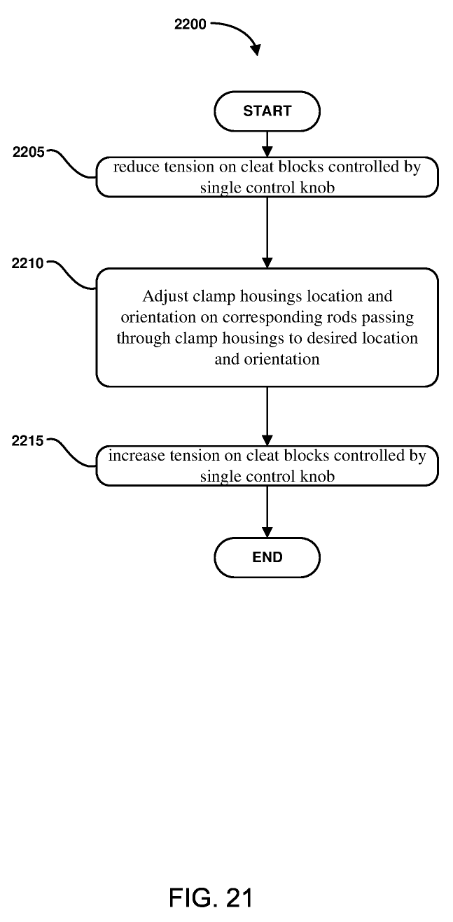

FIG. 21 is a flowchart diagram that illustrates the method operations 2200 performed in adjusting the support arm to a desired position, for implementing embodiments of the present disclosure. The operations illustrated herein are by way of example, as it should be understood that some operations may have sub-operations and in other instances, certain operations described herein may not be included in the illustrated operations. With this in mind, the method and operations 2200 will now be described.

In an operation 2205, the single control knob is loosened to reduce the tension on the control screw and the cleat blocks controlled by the single control knob. In an operation 2210, the clamp housings are adjusted by rotating or sliding or otherwise moving the clamp housings around and/or along a length of the corresponding rods passing through clamp housings to the desired location and orientation. In an operation 2215, the single control knob is tightened to increase tension on the control screw in the cleat blocks controlled by the single control knob which locks the corresponding clamp housings in the desired location orientation. And the method operations can then end.

It should be appreciated that the present disclosure can be implemented in numerous ways, including as a process, an apparatus, a system, or a device. Several inventive embodiments of the present disclosure are described below. Other aspects and advantages of the disclosure will become apparent from the detailed description, taken in conjunction with the accompanying drawings, illustrating by way of example the principles of the disclosure.

Although the foregoing disclosure has been described in some detail for purposes of clarity of understanding, it will be apparent that certain changes and modifications may be practiced within the scope of the appended claims. Accordingly, the present embodiments are to be considered as illustrative and not restrictive, and the disclosure is not to be limited to the details given herein, but may be modified within the scope and equivalents of the appended claims.

What is claimed is:

1. A clamping system comprising:
a single knob coupled to a control screw, the single knob being capable of increasing or decreasing a clamping force on the control screw;
a first clamp housing including:
a first control screw channel, wherein the control screw is disposed in the first screw channel;
a first rod channel capable of receiving a first rod;
a first cleat channel extending into the first rod channel; and
a first cleat block having a first clamping surface capable of entering at least a portion of the first rod channel as the clamping force is increased on the first cleat block.

2. The system of claim 1, wherein the first rod channel is substantially round.

3. The system of claim 1, wherein the first rod channel is substantially perpendicular to the first screw channel.

4. The system of claim 1, wherein increasing the clamping force on the first cleat block through the control screw includes increasing a friction between the first housing and the first rod received in the first rod channel.

5. The system of claim 1, further comprising an alternate rod channel capable of receiving an alternate rod.

6. The system of claim 5, wherein the first rod channel and the alternate rod channel are substantially parallel.

7. The system of claim 5, wherein the first rod channel and the alternate rod channel have non-equal widths.

8. The system of claim 5, wherein the first cleat channel extends into the alternate rod channel and wherein the first cleat block includes an alternate clamping surface capable of entering at least a portion of the alternate rod channel as the clamping force is increased on the first cleat block.

9. The system of claim 8, wherein increasing the clamping force on the first cleat block through the control screw includes increasing a friction between the first housing and the alternate rod received in the alternate rod channel.

10. The system of claim 1, further comprising a second clamp housing including a second control screw channel aligned with the first control screw channel, wherein the control screw is disposed in the second screw channel and wherein increasing the clamping force on the first cleat block through the control screw includes increasing a friction between the first housing and the second housing.

11. The system of claim 10, wherein the second clamp housing further comprising:
a second rod channel capable of receiving a second rod;
a second cleat channel extending into the second rod channel; and
a second cleat block having a clamping surface capable of entering at least a portion of the second rod channel as the single knob increases the clamping force on the second cleat block through the control screw.

12. The system of claim 10, further comprising a friction system disposed between the first clamp housing and the second clamp housing.

13. The system of claim 12, wherein the friction system includes a pair of indexed discs.

14. The system of claim 12, wherein the second clamp housing further includes an optional cavity and wherein the friction system is disposed in the optional cavity.

15. The system of claim 1, further comprising a first spring disposed in the first cleat channel, between the first cleat block and a bottom surface of the first cleat channel.

16. The system of claim 1, wherein the first clamp housing includes an index system including at least one index hole.

17. The system of claim 1, wherein the first cleat block includes raised portion on a bottom surface of the first cleat block.

18. A support arm system comprising:
a support arm;
a base clamp coupled to a first end of the support arm;
a rod coupled to a second end of the support arm, the first end being opposite from the second end;
a rod clamping system disposed on the rod, the rod clamping system including:
a single knob coupled to a control screw, the single knob being capable of increasing or decreasing a clamping force on the control screw;
a first clamp housing including:
a first control screw channel, wherein the control screw is disposed in the first screw channel;
a first rod channel capable of receiving the rod;
a first cleat channel extending into the first rod channel; and
a first cleat block having a first clamping surface capable of entering at least a portion of the first rod channel as the clamping force is increased on the first cleat block.

19. A method of clamping a rod in a clamp housing comprising:
disposing the rod in a rod channel in the clamp housing, the clamp housing including:
a first control screw channel, wherein a control screw is disposed in the first screw channel;
a first rod channel capable of receiving the rod;
a first cleat channel extending into the first rod channel; and
a first cleat block; and
a single knob coupled to the control screw, the single knob being capable of increasing or decreasing a clamping force on the control screw; and
increasing a clamping force with the single knob, including pushing a first clamping surface of the cleat block into at least a portion of the first rod channel and increasing friction between the rod and the first clamp housing.
